(19) United States
(12) Patent Application Publication
(10) Pub. No.: US 2026/0013803 A1
(43) Pub. Date: Jan. 15, 2026

(54) ADAPTIVE ALARMS BASED ON RESCUER CHARACTERISTICS

(71) Applicant: Stryker Corporation, Portage, MI (US)

(72) Inventors: Sophie Francis, Carnation, WA (US); Dara Friedman, Redmond, WA (US); McKenzie Oster, Kirkland, WA (US); Kincade Moran, Kirkland, WA (US); Nolan Frank Dost, Woodinville, WA (US); Franco Gopalakrishnan, Bothell, WA (US); Megan Cote, Redmond, WA (US); Tyler Marshall, Bothell, WA (US); Taylor Christian Sears, Bellingham, WA (US); Tom Hiblar, Lake Stevens, WA (US); Trenton Roeber, Carnation, WA (US); Kai Nellermoe, Kirkland, WA (US)

(73) Assignee: Stryker Corporation, Portage, MI (US)

(21) Appl. No.: 19/266,366

(22) Filed: Jul. 11, 2025

Related U.S. Application Data
(60) Provisional application No. 63/670,020, filed on Jul. 11, 2024.

(51) Int. Cl. A61B 5/00; A61B 5/0205; A61B 5/024; A61B 5/0245; A61B 5/16; A61B 5/364; G08B 21/18

(52) U.S. Cl. CPC A61B 5/746; A61B 5/0006; A61B 5/0205; A61B 5/02438; A61B 5/02455; A61B 5/165; A61B 5/364; A61B 5/4848; A61B 5/681; A61B 5/7405; A61B 2503/20; G08B 21/182

(57) ABSTRACT

Techniques for administering therapy and monitoring a subject based on communication between medical devices are described. An example method includes customizing, by a medical device, alarms (e.g., setting alarm priority and/or thresholds) of the medical device based on communication from a rescuer device. In some instances, the medical device determines (e.g., infers) a characteristic (e.g., medical condition) of a rescuer based on treatment data of the rescuer in an emergency scene, and customizes alarms suitable for the rescuer's condition.

### CROSS-REFERENCE TO RELATED APPLICATION

This application claims priority to U.S. Provisional App. No. 63/670,020, which was filed on Jul. 11, 2024 and is incorporated by reference herein in its entirety.

### BACKGROUND

Medical devices can be used to facilitate patient monitoring, facilitate patient treatments, or a combination of both. Many medical devices are also configured to output alarms that are intended to alert a caretaker with respect to a medical condition of the patient. At an emergency scene, several medical devices may be used contemporaneously to monitor and/or treat a patient, and each of these medical devices can potentially output several different alarms. In these scenarios, caretakers can be exposed to an excessive number of alarms, which can result in desensitization to the alarms and an increased rate of missed or ignored alarms, which renders the alarms ineffective. This type of sensory overload is also referred to as “alarm fatigue.”

### BRIEF DESCRIPTION OF THE DRAWINGS

FIG. 1A illustrates an example of a medical device at an emergency scene where a patient is located, the medical device being configured to output an alarm with a type that is customized to a characteristic of a rescuer.

FIG. 1B illustrates an example of a medical device at an emergency scene where a patient is located, the medical device being configured to output an alarm with a type that is customized according to a type of treatment provided by a rescuer to the patient.

FIG. 2 illustrates a particular example of a rescuer device configured to determine that a rescuer is likely patient is likely performing treatment to a patient, and to customize a set of alarms of the medical device to obtain a customized set of alarms tailored to the determined treatment.

FIG. 3 illustrates an example process for determining a parameter of a rescuer from context of an emergency scene where the rescuer and a patient are located, and customizing a set of alarms of a medical device to the parameter of the rescuer in order to output an alarm that is customized to the rescuer.

FIG. 4 illustrates an example process for determining, from treatment data determined by a medical device, a parameter of a rescuer to which a type of treatment administered by the rescuer corresponds, and customizing a set of alarms of the medical device to the type of treatment to output an alarm that is customized to the type of treatment.

FIG. 5 illustrates example techniques in a process for determining a characteristic of a treatment of a rescuer from rescuer data, and context of an emergency scene where the rescuer is located.

FIG. 6 illustrates example techniques in a process for customizing a set of alarms.

FIG. 7 illustrates an example process for managing alarms of a medical device in the context of controlling an additional medical device(s) in a vicinity of the medical device.

FIG. 8 illustrates an example process for managing alarms of a medical device based on a medication administered to a patient at an emergency scene.

FIG. 9 illustrates an example process for preconfiguring the alarms of a medical device and simulating the alarms via an alarm and boundary configuration user interface presented on a user device.

FIG. 10 illustrates an example of an external defibrillator configured to perform various functions described herein.

### DETAILED DESCRIPTION

Various implementations described herein relate to techniques for managing alarms of a medical device by adapting the alarms and customizing the alarms to a treatment administered by a rescuer and to a patient located at an emergency scene. In some examples, the rescuer arrives at the emergency scene with a medical device that is to be used for monitoring and/or treating the patient. In some scenarios, the rescuer, upon arriving at the emergency scene, has no prior knowledge of who the patient is, let alone the patient's medical history. Moreover, at the time of arrival, the rescuer may not know the current medical condition of the patient and/or what type of treatments might help the patient.

In particular, medical devices utilize alarms to alert rescuers of various aspects related to operations of the medical devices and/or treatments for which the medical devices are utilized. However, if an alarm of a medical device is not recognized by the rescuer, the medical device may be unable to safely perform its functions. In some examples, the emergency scene is at a public location with many bystanders, ambient noise, other alarms, device noises, device actions, and/or distractions that can potentially detract the rescuer's attention from the patient in need of medical care. In some examples, multiple medical devices are managing care of the same patient, each with their own set of alarms that are intended to alert rescuers of potential danger with respect to a medical condition of the patient. These types of alarms can cause problems for the rescuer at the emergency scene. The alarms may cause overlooking of the alarm by the rescuer, confusion between the alarm and other alarms of the medical devices or other medical devices, apathy by the rescuer of the alarm, and so on, or any combination thereof. Thus, it is desirable for the alarm to be customized from among alarms of different types corresponding to the role of the rescuer and/or treatment related aspects of the emergency scene.

The techniques, devices, and systems described herein address the above mentioned, and other problems, by customizing alarms of a medical device, such as an external monitor-defibrillator. The alarms may be adapted, for instance, so that the alarms are customized to characteristics of the rescuer, thereby rendering the alarms more effective in alerting the rescuer. According to various implementations of the present disclosure, a medical device is configured to output an alarm with a type that is customized to a characteristic of the rescuer and/or a characteristic of a treatment administered by the rescuer. For example, the type of the alarm may be determined from a physiological characteristic of the rescuer. In some implementations, the medical device may include a transceivers configured to receive a communication from a rescuer device of the rescuer. The rescuer device may be worn, and/or in contact with, the rescuer. In some implementations, the rescuer device includes, and/or is communicatively coupled to, a sensor. The sensor may generate rescuer data indicative of the physiological parameter. The rescuer data may represent a physiological parameter of the rescuer. For instance, the physiological parameter may include a heart rate or another indicator of stress. The communication may include the rescuer data. In response to receiving the rescuer data, the medical device may determine the type of the alarm and output the alarm with the type to alert the rescuer.

Alternatively or additionally, the techniques, devices, and systems described herein address the above-mentioned problems and other problems by adapting alarms of a medical device so that the alarms are customized to a treatment of a patient, thereby rendering the alarms more effective in alerting the rescuer providing the treatment. In some implementations, the medical device can generate and/or input rescuer data reflect the treatment provided by the rescuer.

To illustrate, consider an example in which the treatment data includes movement data of the rescuer data. In this example, a rescuer device worn by the rescuer may include a sensor that generates sensor data, which may be utilized by the rescuer device and/or the medical device to generate the treatment data. In this example, the sensor of the rescuer device includes a motion sensor (e.g., accelerometer) utilized to generate the sensor data, which may include the movement data. The movement data may identify cardiopulmonary resuscitation (CPR) related data, reflecting the treatment provided by the rescuer being CPR treatment. In various implementations, an alarm, such as a CPR based alarm associated with cardiac behavior of the patient pertaining to the CPR treatment, may be deprioritized over other alarms not pertaining to the CPR treatment (e.g., the other alarm(s) potentially being of relatively greater use to the rescuer since the rescuer already providing the CPR treatment being aware of the cardiac behavior of the patient but possibly not aware of another patient-related condition(s)). In such an instance or another instance, the sensor may include a heart rate sensor, a haptic sensor, an air flow sensor, etc., utilized to generate the sensor data, which may include movement data, heart rate data, air flow data, etc. The data (e.g., the movement data, the heart rate data, air flow data, etc.), reflecting the treatment provided by the rescuer being CPR treatment. In various implementations, an alarm, such as a movement-based alert associated with the CPR treatment for cardiac behavior of the patient may be controlled to reduce alarm fatigue of the rescuer.

In such an example or another example, a limit (e.g., a high limit and/or a low limit) associated with an alarm may be customized by changing a threshold used for generating the alarm. The alarm may have a customized characteristic for a volume, a pressure, a repetition, an escalation, a de-escalation, a trigger, a cut-off, and so on, or any combination thereof. The customized alarm may be better tailored to the characteristic of the rescuer and/or the treatment administered thereby, which renders the alarm of the medical device more effective in assisting the rescuer at the emergency scene, leading to an improved patient outcome. For example, the alarm being deprioritized may be disabled or muted in order to enable the rescuer to direct their attention to the treatment. Alternatively or additionally, the customized alarm may be more or less sensitive to a certain medical condition to alert the rescuer only when the rescuer needs to be alerted. The customized alarm may be generated by refraining from alerting the rescuer at times when it is unnecessary to do so. In some examples, an alarm type of the alarm includes a CPR-based alarm type corresponding to the chest compressions, the alarm instructing the rescuer to change a frequency, a depth, a location, or a duty cycle of the chest compressions.

It is to be appreciated that, while several of the examples described herein pertain to monitor-defibrillators, the techniques described herein may be implemented with respect to other types of medical devices. Moreover, while several of the examples described herein pertain to an emergency scene that is outside of a hospital setting, the techniques described herein may be equally useful for in-hospital settings, such as when a patient initially arrives at a hospital and is taken to an emergency room.

Implementations of the present disclosure are directed to improvements in the technical field of medical devices. With conventional medical devices, caretakers, such as rescuers who arrive at an emergency scene where a patient is located, may experience alarm fatigue, disorientation, inundation, over stimulation, apathy, indifference, confusion, and so on, or any combination thereof, due to their exposure to an excessive number of alarms output by the medical devices. The techniques, devices, and systems described herein mitigate alarm related problems by adapting the alarms of a medical device so that the alarms are customized to a rescuer who is located at an emergency scene and/or a treatment provided thereby. The customized alarms may render the alarms more effective in alerting a caretaker (e.g., the rescuer) with respect to a medical condition of the patient, which leads to improved patient outcomes. By improving patient outcomes, likelihoods of providing successful treatment and saving lives of the patients in some circumstances, including highly stressful, high risk, and/or time sensitive circumstances, may be improved.

Various examples will now be described with reference to the accompanying drawings.

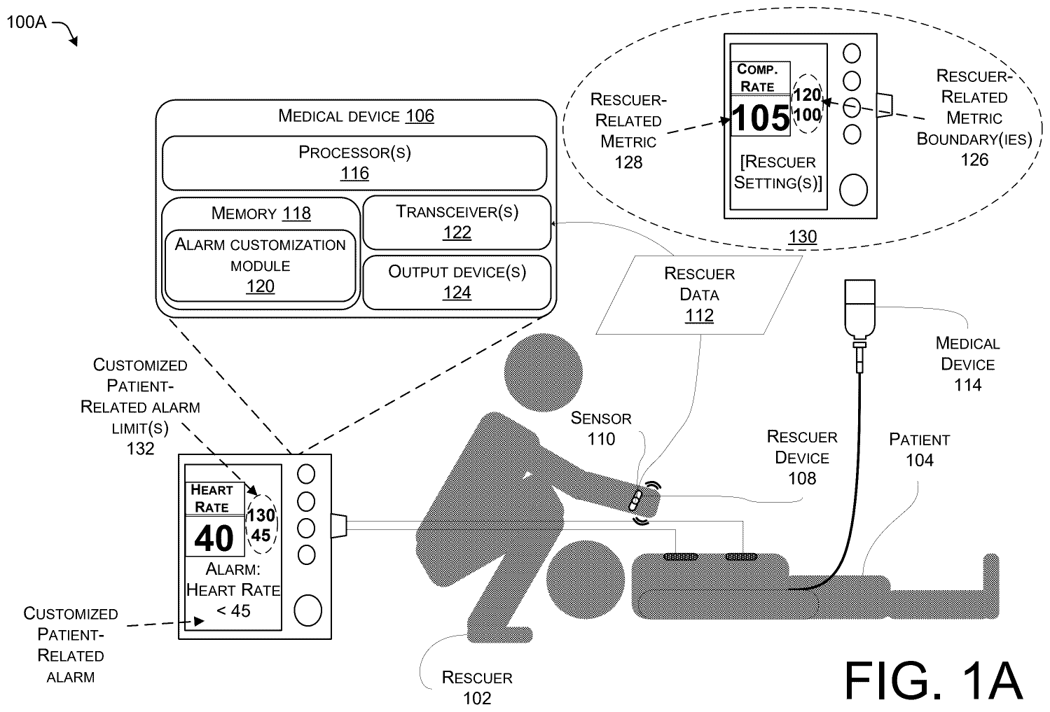

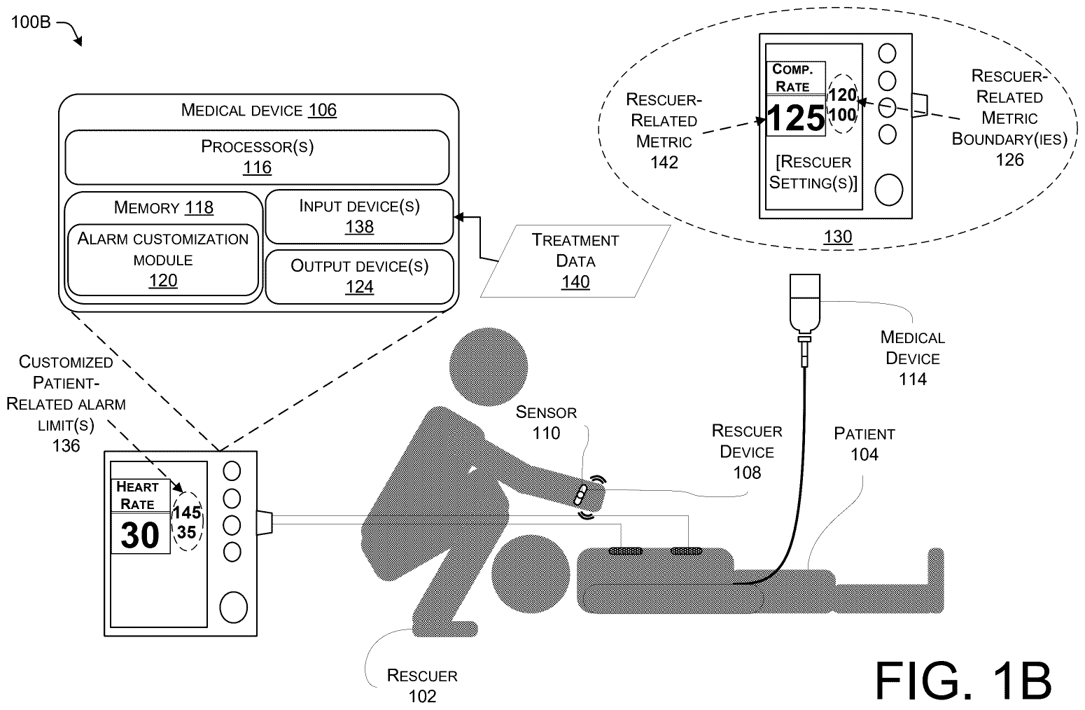

FIG. 1A illustrates a medical device at an emergency scene 100A where a patient is located, the medical device configured to output an alarm with a type that is customized to a characteristic of a rescuer. The emergency scene 100A, in some implementations, is outside of a clinical environment, such as a public area that is located remotely from a hospital or other managed care facility. In some cases, the emergency scene 100A is within a clinical environment, such as within an emergency room at a hospital. In various implementations, the rescuer, such as a rescuer 102, is monitoring and/or treating the patient, such as a patient 104. For examples, the rescuer 102 and/or the patient 104 are located at the emergency scene 100A. In some instances, the rescuer 102 is an emergency medical technician (EMT) who has arrived at the emergency scene 100A to monitor and/or treat a medical condition of the patient 104 using a medical device(s). For instance, the rescuer 102 can administer treatment using the medical device, such as a medical device 106. In some cases, the patient 104 is experiencing cardiac arrest, a blocked airway, or some other dangerous medical condition.

In some implementations, the rescuer 102 is monitoring and/or treating a medical condition of the patient 104 using the medical device 106. For instance, the medical device 106 is a monitor-defibrillator, a medical imaging device, an ultrasound monitor, a standalone electrocardiogram (ECG) monitor, or another type of medical device.

As shown in FIG. 1A, the rescuer 102 is administering treatment in the emergency scene 100A, in which a rescuer device 108 is located. In some examples, the rescuer device 108 is located within a threshold distance from the rescuer 102. In those or other examples, the rescuer device 108 is a wearable device worn by the rescuer 102. For instance, the rescuer device 108 is a portable computing device, such as smart glasses, a smart watch, a recording device, or any other types of computing device. In some examples, the rescuer device 108 includes a sensor 110 utilized to generate rescuer data 112. In those or other examples, the rescuer device 108 communicates the rescuer data 112 to the medical device 106.

Although FIG. 1A depicts an example rescuer 102 wearing an example rescuer device 108, such as a wearable smart watch, other types of rescuers, and/or other types of devices therefor, are contemplated herein, such as a motion detector, a haptic feedback device, an audio and/or video input device, and/or the like. Accordingly, the rescuer data 112 may indicate any suitable type of physiological parameter of the rescuer 102, such as any of the example physiological parameters described above with reference to the sensor 110.

For example, the rescuer device 108 detects a parameter, such as a physiological parameter, of the rescuer 102. In some cases, the physiological parameter includes a heart rate, a blood pressure, a respiration rate, a temperature, an acceleration, a location, a vocal sound, a blood oxygenation, a pupil movement, and/or any number of any other types of parameters of the rescuer 102. In alternative or additional examples, the parameter of the rescuer 102 includes a physiological parameter, a level of body movement detected by a motion sensor of a wearable device worn by the rescuer, a volume of a vocal sound detected by an audio recorder of the wearable device, or a pitch of the vocal sound. In alternative or additional examples, the parameter of the rescuer 102, for instance, corresponds to a predicted stress level of the rescuer 102. In some cases, the parameter of the rescuer 102 includes a physiological parameter corresponding to a probability of a level of stress of the rescuer 102.

In various examples, the rescuer device 108 includes and/or is communicatively coupled to a sensor 110. For example, the sensor 110 includes at least one of an accelerometer, electrodes, a detection circuit, a force sensor, an ultrasound-based blood pressure sensor, a gas sensor (e.g., a carbon dioxide and/or oxygen sensor), a flowmeter, a microphone, defibrillator pads, a blood pressure cuff, an invasive (e.g., intra-arterial) blood pressure sensor (e.g., including a cannula inserted into the patient 104), a pulse oximetry sensor, a regional oximetry sensor, a thermometer, an ultrasound transducer, a medical imaging device (e.g., an ultrasound imaging device), or the like, or any combination thereof. In some instances, the sensor 110 is configured to detect a physiological parameter(s) of the rescuer 102. Examples of physiological parameter(s) of the rescuer 102 include a movement, a heart rate, a blood pressure, a respiration rate, a temperature, an acceleration, a location, a vocal sound, a pupil movement of the rescuer 102, any other physiological parameters, or any combination thereof. In various cases, the rescuer device 108 outputs the physiological parameter(s) to the medical device 106.

In some examples, an additional medical device 114 may be collocated at the emergency scene 100A with the medical device 106. In the example of FIG. 1A, an additional medical device(s) 114 included in the emergency scene 100A represents a medication-administering device, such as an intravenous fluid (IV) pump. The additional medical device 114 is merely an example, and it is to be appreciated that other types of medical devices 114 may be in a vicinity of the medical device 106 at the emergency scene 100A, such as a monitor-defibrillator, an automated external defibrillator (AED), a smart bag-valve mask, a ventilator, a heart-lung machine, a mechanical chest compression device, and/or the like. Examples of treatments (e.g., therapies) that can be administered by the medical device 106 and/or the additional medical device(s) 114 include defibrillation, pacing, cardioversion, administration of chest compressions, administration of oxygen to the airway of the patient 104, movement of air in the airway of the patient, administration of fluids to the patient 104, extracorporeal membrane oxygenation (ECMO), administration of a medication to the patient 104, and/or the like. In some implementations, the additional medical device(s) 114 is configured to detect a physiological parameter(s) of the patient 104, such as the example physiological parameters described above with respect to the medical device 106.

In some cases, the medical device 106 includes a display, a speaker, or haptic feedback device that conveys the physiological parameter(s) of the patient 104 to the rescuer 102. In the example of FIG. 1A, the medical device 106 is outputting a parameter, such as a movement parameter. Any number of parameters (e.g., the movement parameter, a heart rate parameter, a carbon dioxide parameter, such as an end tidal carbon dioxide (EtCO₂) parameter, and so on, or any combination thereof) are included, in alternative or additional examples. For instance, the medical device 106 is connected to a sensor(s) utilized to detect a physiological parameter(s) of the patient 104.

In some examples, the medical device 106 includes and/or is communicatively coupled to the sensor(s) utilized to detect any number of various types of physiological parameters of the patient 104. For instance, the physiological parameter(s) of the patient 104 that is detected includes an ECG, an impedance (e.g., a transthoracic impedance), a force administered to the patient 104, a blood pressure, an airway parameter (e.g., a partial pressure of carbon dioxide, a partial pressure of oxygen, a capnograph, an end tidal gas parameter, a flow rate, etc.), a blood oxygenation (e.g., a pulse oximetry value, a regional oximetry value, etc.), an electroencephalogram (EEG), a temperature, a heart sound, a blood flow rate, a physiological geometry (e.g., a shape of a blood vessel, an inner ear shape, etc.), a heart rate, a pulse rate, or the like. For example, the sensor(s) utilized to detect a physiological parameter(s) of the patient 104 includes at least one of a movement sensor (e.g., an accelerometer), electrodes, a detection circuit, defibrillator pads, a force sensor, a blood pressure cuff, an ultrasound-based blood pressure sensor, an invasive (e.g., intra-arterial) blood pressure sensor (e.g., including a cannula inserted into the patient 104), a gas sensor (e.g., a carbon dioxide and/or oxygen sensor), a flowmeter, a pulse oximetry sensor a regional oximetry sensor, a thermometer, a microphone, an ultrasound transducer, a medical imaging device (e.g., an ultrasound imaging device), or the like.

FIG. 1A illustrates example components of the medical device 106. For example, the medical device 106 includes a processor(s) 116, such as a central processing unit(s) (CPU(s)), a graphics processing unit(s) (GPU(s)), both CPU(s) and GPU(s), or another processing unit or component known in the art. The processor(s) 116 is operably connected to memory 118. In various implementations, the memory 118 is volatile (such as random access memory (RAM)), non-volatile (such as read only memory (ROM), flash memory, etc.) or some combination of the two. The memory 118 stores instructions that, when executed by the processor(s) 116, cause the processor(s) 116 to perform various operations described herein. In various examples, the memory 118 stores methods, threads, processes, applications, objects, modules, any other sort of executable instruction, or a combination thereof. An examples depicted in FIG. 1A includes an alarm customization module 120 that, when executed by the processor(s) 116, causes the processor(s) 116 to, among other things, determine a characteristic of the rescuer 102 by analyzing the rescuer data 112 (e.g., data received via a transceiver(s) 122, as discussed below in further detail), and customize a set of alarms to the determined rescuer characteristic to obtain a customized set of alarms that is tailored to the rescuer 102. In various examples, the rescuer device 108 includes any number of components being similar to, or different from, any components of the medical device 106.

As used herein, the term “module,” and its equivalents, refers to data including instructions that, when executed by a processor(s), cause the processor(s) to perform an operation(s). In some cases, the memory 118 stores files, databases, or a combination thereof. For example, the medical device 106 and/or the rescuer device 108 collects physiological parameter data (e.g., CO₂ data, heart rate data, etc.) associated with the rescuer 102 during use of the medical device 106 and/or the rescuer device 108, and this and other data may be stored, at least temporarily, in the memory 118 and/or in a memory of the rescuer device 108. In some examples, the memory 118 includes RAM, ROM, electrically erasable programmable read-only memory (EEPROM), flash memory, or any other memory technology. In some examples, the memory 118 includes CD-ROMs, digital versatile discs (DVDs), content-addressable memory (CAM), and/or other optical storage, magnetic cassettes, magnetic tape, magnetic disk storage and/or other magnetic storage devices, and/or any other medium (e.g., non-transitory computer-readable medium) which can be used to store the desired information and which can be accessed by the processor(s) 116.

In the example of FIG. 1A, the medical device 106 is shown as having a transceiver(s) 122. The transceiver(s) 122 (e.g., a wireless radio, antenna, or the like) may be configured to communicating wirelessly with another device ((s) at the emergency scene 100B, such as the rescuer device 108 of the rescuer 102, and/or the additional medical device(s) 114 described above with reference to FIG. 1A. The transceiver(s) 122 may include any sort of wireless transceivers capable of engaging in wireless communication (e.g., radio frequency (RF) communication), such as WI-FI®, WIGIG®, WIMAX®, BLUETOOTH®, near-field communication (NFC), radio frequency identification (RFID), or infrared communication.

In some cases, the monitoring and/or treatment of the patient 104 is optimized by communication between the medical device 106 and another device(s), such as the rescuer device 108 and/or a patient device (e.g., a device associated with, and/or worn by, the patient 104, the patient device including components that are similar to, or different from the medical device 106 and/or the rescuer device 108, and/or being similar to, or different from, the medical device 106 and/or the rescuer device 108). In particular examples, the transceiver(s) 122 is configured to receive, from the rescuer device 108, the rescuer data 112 indicative of a physiological parameter(s) of the rescuer 102, such as one or more of the physiological parameter(s) described above with reference to the sensor 110.

To exchange data, the medical device 106 and the rescuer device 108, an additional medical device(s) 114, and/or the patient device are configured to establish and/or communicate via a communication channel. As used herein, the term “communication channel,” and its equivalents, may refer to a medium over which a first endpoint (e.g., a sender) transmits information to a second endpoint(s) (e.g., receivers). Examples of communication channels include wired connections, such as Ethernet or fiber optic paths, as well as wireless connections, such as Institute of Electronics and Electrical Engineers (IEEE) (e.g., WI-FI, BLUETOOTH, etc.) or 3^{rd} Generation Partnership Program (3GPP) (e.g., Long Term Evolution (LTE), New Radio (NR), etc.) connections. As used herein, the term “endpoint,” and its equivalents, may refer to an entity that is configured to transmit and/or receive data. Examples of endpoints include user equipment (UE) (e.g., mobile phones, tablet computers, etc.), computers, base stations, access points (Aps), servers, compute nodes, medical devices, Internet of Things (IoT) devices, and the like.

In some implementations, a communication channel between the medical device 106 and the rescuer device 108 is established when the medical device 106 and the rescuer device 108 are paired. A similar pairing procedure may be utilized between the medical device 106 and another device (e.g., the rescuer device 108, an additional medical device(s) 114, the patient device, etc.), and/or between the rescuer device 108 and another device (e.g., an additional medical device(s) 114, the patient device, etc.). In particular cases, two devices (e.g., the medical device 106 and the rescuer device 108) refrain from sharing substantive data (e.g., the rescuer data 112, physiological metrics, reports about the rescuer 102, instructions regarding treatment administered by the rescuer 102, etc.) until the two devices are paired. As used herein, the term “paired,” and its equivalents, may refer to a state of multiple devices that have a shared link key that enables each device to cryptographically authenticate data it receives from any other device among the multiple devices. In some examples, the medical device 106 may send, to the rescuer device 108, via the transceiver(s) 122, authentication data for authenticating the medical device 106 prior to receiving the rescuer data 112. In this manner, for instance, the rescuer device 108 refrains from sending the rescuer data 112 to the medical device 106 until the medical device 106 authenticates with the rescuer device 108.

Various mechanisms can be utilized to pair two devices, such as the medical device 106 and the rescuer device 108. For example, automated pairing involves the exchange of data and/or pairing requests/responses between the devices 106 and 108. As another example, the medical device 106 and/or the rescuer device 108 receives an input signal from an operator (e.g., the rescuer 102) that selects the rescuer device 108 and/or the medical device 106, respectively, as a device to pair with. In some cases, the medical device 106 detects an alternative signal (e.g., a flashing light pattern) from the rescuer device 108 that is indicated in a pairing request, or vice versa. In some cases, the medical device 106 and the rescuer device 108 are paired, at least in part, based on signaling to and/or from an intermediary device (not illustrated in FIG. 1A). In a particular example, two or more devices can be brought into proximity to (e.g., into contact with) each other in order to facilitating pairing the medical device 106 with the rescuer device 108. For example, a “tap-to-pair” functionality may allow a user to bring the rescuer device 108 (or a component thereof) into close proximity to (e.g., into contact with) the medical device 106 (or a component thereof), or vice versa, and a short-range wireless protocol, such as BLUETOOTH, near-field communication (NFC), or the like, may be used to detect that the devices 106 and 108 are within a threshold distance of each other, and, in response, the devices 106 and 108 may be paired. In some cases, an intermediary device, such as a phone, may be brought into close proximity to (e.g., into contact with) the medical device 106 and/or the patent device 108 (e.g., by touching the intermediary device to both devices 106 and 108 sequentially), and a short-range wireless protocol may be used to detect these proximity events involving the intermediary device, and, in response, the devices 106 and 108 may be paired. Similar pairing techniques are utilized, in some cases, between the devices 106 and/or 108 and/or any other devices, such as the patient device.

In particular cases, a first paired device encrypts data prior to transmitting the data to a second paired device, and the second paired device restores the original data by decrypting the encrypted data. As used herein, the term “encrypt,” and its equivalents, refers to a process of translating data from one format (e.g., an unencoded format) into an encoded format. In various cases, the encoded format is referred to as “ciphertext.” Unencoded data, which has not been encrypted, may be referred to as being in “plaintext.” In various examples, an entity encrypts data using at least one encryption key. An encryption key is a parameter that defines the translation of data from the one format into the encoded format. As used herein, the term “decrypt,” and its equivalents, refers to a process of translating data from an encoded format into another format (e.g., an unencoded format), such as a plaintext format. In various examples, an entity encrypts data using at least one decryption key. A decryption key is a parameter that defines the translation of data from the encoded format into the other format. A link key, for example, is an encryption and/or decryption key.

Various cryptographic techniques can be utilized in accordance with the features described in this disclosure. For example, data can be encrypted and decrypted via a symmetric key, wherein the encryption key and the decryption key are equivalent. In some cases, data can be encrypted and decrypted via asymmetric keys, wherein the encryption key and the decryption key are different. Cryptographic hash functions (CHFs) are examples of cryptographic techniques. Examples of cryptographic techniques include the Data Encryption Standard (DES), Advanced Encryption Standard (AES), Elliptic Curve Cryptography (ECC), Rivest-Shamir-Adleman (RSA), Secure Hash Algorithm (SHA)-1, SHA-2, SHA-3, BLAKE, BLAKE2, BLAKE3, WHIRLPOOL, MD2, MD4, MD5, MD6, Temporal Key Integrity Protocol (TKIP), Rivest cipher 4 (RC4), variably modified permutation composition (VMPC), blowfish, Twofish, Threefish, Tiny Encryption Algorithm (TEA), Extended TEA (XTEA), Corrected Block TEA (XXTEA), Diffie-Hellman exchange (DHE), elliptic curve DHE, supersingular isogeny Diffie-Hellman (SIDH) key exchange, and so on. Any suitable encryption or decryption technique can be used in accordance with implementations of this disclosure. Accordingly, in some examples, the rescuer data 112 is encrypted and transmitted to the medical device 106, and the processor(s) 116 is configured to decrypt the rescuer data 112 prior to customizing a set of alarms.

In the example of FIG. 1A, the transceiver(s) 122 receives, from the rescuer device 108, the rescuer data 112 indicative of a parameter(s) (e.g., a physiological parameter(s)) of the rescuer 102, and the processor(s) 116 (e.g., via execution of the alarm customization module 120) customizes a set of alarms associated with the medical device 106 and/or the rescuer device 108 to obtain a customized set of alarms that is customized to the physiological parameter(s) of the rescuer 102. As used herein, the term “set of alarms” may refer to data representing one or more alarm types (e.g., a CPR-based alarm type), thresholds, alarm escalations, or the like. Consider an example where the rescuer data 112 indicates a parameter(s) (e.g., a movement parameter(s), a heart rate, etc., or any combination thereof) of the rescuer 102, the processor(s) 116 identifies the parameter(s) of the rescuer 102, and possibly a boundary(ies) associated with the parameter(s). For example, the processor(s) 116 identifies the parameter(s) and the parameter boundary(ies), and changes at least one of the customized set of alarm(s) and/or a threshold(s) used for generating a cardiovascular-related alarm, such as a heart rate alarm associated with the patient 104. The processor(s) 116, in some instances, changes the alarm(s) and/or the threshold(s) based on the parameter(s) and/or the boundary(ies). The boundary(ies), for example, indicate a point(s), and/or act as a trigger(s), for adjusting the alarm(s) and/or the threshold(s), based on the parameter(s) of the rescuer exceeding the boundary(ies) (e.g., going below a low boundary, going above a high boundary, etc.).

The parameter boundary(ies) and the parameter(s) of the rescuer 102 being utilized to change the at least one of the customized set of alarm(s) and/or the threshold(s) is shown in FIG. 1A as an rescuer-related metric boundary(ies) 126 and an rescuer-related metric 128, respectively. That is, the rescuer-related metric boundary(ies) 126 includes a high boundary associated with the rescuer-related boundary that is changed from, say, an initial value (e.g., a default value) of 115 compressions (or “comp's”) per minute to 120 compressions per minute, and/or a low boundary associated with the rescuer-related boundary that is changed from, say, an initial value (e.g., a default value) of 105 compressions per minute to 100 compressions per minute.

In some implementations, the medical device 106 identifies, via a mode (e.g., a rescuer characteristics-based mode) 130 associated with a rescuer setting(s), the boundary(ies) 126, based on various types of data identified by the medical device 106. For example, the medical device 106 identifies, via the mode 130, the boundary(ies) 126, based on user input provided to the medical device 106 (e.g., via a touch-sensitive display, keypad, etc.). In those or other examples, the medical device 106 identifies the boundary(ies) 126 via trained artificial intelligence (AI) model analysis (e.g., and/or trained AI model output), alternatively or in addition to the user input. Alternatively or additionally to utilizing the mode 130 to manage (e.g., output, control, etc., or any combination thereof) the alarm(s) and/or control the alarm threshold(s), the rescuer-related parameter(s) are utilized to adjust the alarm(s) and/or the alarm threshold(s) during any type of operation performed by the medical device 106, and/or for any mode (e.g., an operating mode, etc.) of the medical device 106.

In some examples, the medical device 106 possibly outputs various types of rescuer-related data and/or setting(s). For instance, the medical device 106 outputs (e.g., presents) the boundary(ies) 126 and/or the rescuer-related metric 128, while the medical device 106 operates in the rescuer characteristics-based mode 130. Alternatively or additionally, the medical device 106, for instance, outputs any other rescuer-related data, such as while the medical device 106 is operating in the rescuer characteristics-based mode 130. The rescuer characteristics-based mode 130 is utilized, for instance, by the rescuer 102 to set the boundary(ies) 126.

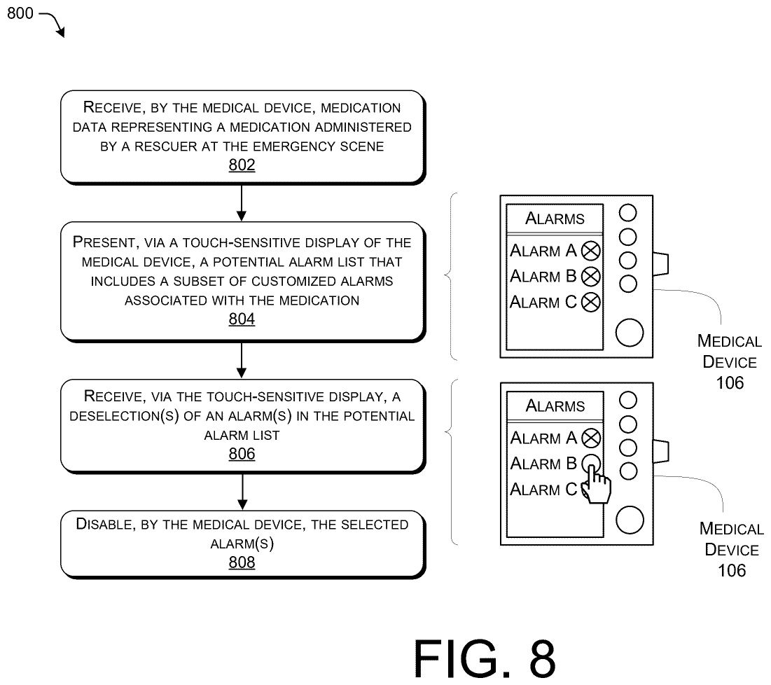

In various examples, the rescuer characteristics-based mode 130 is activated in various ways for setting any of the boundary(ies) associated with any of the rescuer parameter(s). For instance, the mode 130 is activated via selection of an alarm, such as the “Alarm(s) A, B, or C,” as discussed below in further detail with reference to FIG. 8. In various instances, the mode 130 is utilized to set boundary(ies) for a number of parameter(s) relevant to adjusting (e.g., automatically adjusting, such as without requiring input by the rescuer 102 during treatment administered by the rescuer 102) the alarm(s) and/or the alarm threshold(s).

In various implementations, the medical device 106 and/or the rescuer device 108 perform analysis, and/or exchange communications, to manage the boundary(ies) 126 and/or any type(s) of data (e.g., the rescuer data 112, the treatment data 140, as discussed below in further detail with reference to FIG. 1B, and so on, or any combination thereof) associated with the rescuer 102 and/or the treatment administered thereby. Alternatively or additionally, the medical device 106 and/or the rescuer device 108 perform analysis, and/or exchange communications, to manage the alarm(s) and/or the alarm threshold(s). For instance, the medical device 106 and/or the rescuer device 108 utilize user input and/or the trained AI model analysis (e.g., trained AI model output) to manage the alarm(s) and/or the alarm threshold(s). By way of example, the user input, and/or analysis performed by a trained AI model(s), as discussed below in further detail, are utilized to manage the alarm(s) and/or the alarm threshold(s) based on the boundary(ies) 126, and/or the data (e.g., the parameter(s), such as in the rescuer data 112) associated with the rescuer 102 and/or the treatment administered thereby.

After obtaining the rescuer-related metric boundary(ies) 126, the processor(s) 116 is configured to determine how to manage (e.g., identify whether to generate) an alarm(s) of the customized set of alarms based on the parameter(s) of the rescuer 102. For example, the sensor 110 detects the rescuer-related metric 128, such as the parameter(s) of the rescuer 102. In such an example or another example, the rescuer-related metric 128 includes a compression rate, and the processor(s) 116 receives the rescuer-related metric 128 via the sensor 110 and compares the rescuer-related metric 128 to the boundary(ies) (e.g., the rescuer-related metric boundary(ies)) 126.

In some implementations, a customized patient-related alarm limit(s) 132 and/or a customized patient-related alarm 134 are managed (e.g., adjusted) based on the boundary(ies) 126 and the rescuer-related metric 128 are utilized by the medical device 106. By way of example, in response to comparing the metric 128 to the boundary(ies) 126, the processor(s) 116 adjusts a limit(s) of the alarm limit(s), such as the customized patient-related alarm limit(s) 132. In such an example or another example, in response to comparing the metric 128 to the boundary(ies) 126, the processor(s) 116 generates, or refrains from generating, an alarm(s) of the customized set of alarms, such as the customized patient-related alarm 134.

For example, if the accelerometer indicates that the rescuer-related metric 128, such as the compression rate, is 105 compressions per minute, the processor(s) 116 determines that this compression rate is greater than a rescuer-related metric boundary 126 (e.g., the low boundary of 100 compressions per minute), and less than a rescuer-related metric boundary 126 (e.g., the high boundary of 120 compressions per minute); and the processor(s) 116, in some cases, generates a rescuer-related flag associated with, and/or indicating, the compression rate is between 100-120 compressions per minute.

In various implementations, based on the alarm-oriented rescuer-related flag, and/or based on the rescuer-related metric 128 (e.g., the compression rate) of the rescuer 102 identified as being between the high boundary and the low boundary, the customized patient-related alarm limit(s) 132 is set to have a high limit associated with the heart rate alarm of the patient 104 that is changed from, say, 100 beats per minute (bpm) to 130 bpm, and/or a low limit associated with the heart rate alarm of the patient 104 that is changed from, say, 60 bpm to 45 bpm. In other words, the limit(s) associated with the heart rate alarm for the patient 104 is changed from a relatively narrower range to a relatively wider range in order to decrease the sensitivity of the heart rate alarm associated with the patient 104, or vice versa in order to increase the sensitivity of the heart rate alarm associated with the patient 104.

In various implementations, the processor(s) 116 generates, or refrains from generating, an alarm(s) associated with the medical device 106 and/or the rescuer device 108. For instance, the alarm(s) is the heart rate alarm associated with the patient 104 in this scenario. The processor(s) 116, in some examples, also causes, or refrains from causing, the alarm(s) to be output via an output device(s) 124. In those or other examples, the processor(s) 116 transmits, or refrains from transmitting, a signal to the rescuer device 108. For instance, the signal associated with the alarm is transmitted, or not, based on a signal with the parameter of the rescuer 102 that is received from the rescuer device 108. In those or other examples, such as for instance with the alarm-related signal being transmitted by the medical device 106, the rescuer device 108 receives the signal associated with the alarm(s) and causes the alarm(s) to be output via an output device(s) of the rescuer device 108. The output device(s) of the rescuer device 108, in various cases, is a similar type, or a different type, than the output device(s) 124.

The output device(s), as represented by the output device(s) 124, outputting the alarm(s) (e.g., the customized patient-related alarm 134) is shown, for example, in FIG. 1A. In such a case in which the alarm rate of the patient is 40 bpm, the customized patient-related alarm 134 is output (e.g., visually presented) by the output device(s) 124. In some examples, the customized patient-related alarm 134 is in the form of a visual alarm (e.g., “Advisory: Heart Rate <45) presented on a display of the medical device 106 and an audible alarm (e.g., a siren, a periodic “beep” sound, etc.) output via a speaker(s) of the medical device 106. Of course, the alarm may be output in other ways, such as a flashing LED(s), a vibration of a haptic actuator(s), and/or the like. By customizing the alarm(s) to the physiological parameter(s) of the rescuer 102 indicated by the rescuer data 112, the alarm(s) is rendered more effective in alerting the rescuer 102, only as necessary and/or desired, as to a current medical condition (e.g., a cardiovascular problem) of the patient 104. The output device(s) of the rescuer device 108 are managed, for example, in a similar way as, or a different way from, the output devices) 124.

In various examples, the alarm(s) includes any of various types of alarms. For instance, the medical device 106 (e.g., the processor(s) 116) and/or the rescuer device 108 causes the alarm to be output by causing a device (e.g., the medical device 106 and/or the rescuer device 108) to output a projection indicating the alarm, causing the medical device 106 and/or the rescuer device 108 to output a hologram indicating the alarm, and/or causing the medical device 106 and/or the rescuer device 108 to output an augmented reality (AR) alarm.

FIG. 1B illustrates an emergency scene 100B where a patient is located, the medical device configured to output an alarm with a type that is customized to a characteristic of a rescuer. In some implementations, the emergency scene 100B may be a same emergency scene as, or a different emergency scene than, the emergency scene 100A described above with reference to FIG. 1A. In various implementations, a rescuer 102 is monitoring and/or treating a patient 104 located at the emergency scene 100B. For instance, the rescuer 102 is an EMT who has arrived at the emergency scene 100B to monitor and/or treat a medical condition of the patient 104 using medical device(s), such as the medical device 106 introduced in FIG. 1A and/or another medical device being the same types as, or a different type from, the medical device 106.

In some examples, the medical device 106, being collocated in the emergency scene 100B along with the additional medical device(s) 114, includes the processor(s) 116, the memory 118, the alarm customization module 120, the output device(s) 124. In those or other examples, the medical device 106 possibly includes the transceiver(s) 122. The processor(s) 116, in some instances, manages a customized alarm limit(s) 136. The alarm limit(s) 136, a rescuer-related metric boundary(ies) (e.g., the rescuer-related metric boundary(ies) 126 identified in any of various ways, such as via the mode 130, as discussed above with reference to FIG. 1A, and/or another rescuer-related metric boundary(ies)), and/or a rescuer-related metric (e.g., the rescuer-related metric 142, as discussed below in further detail) are utilized to control the medical device to refrain from outputting an alarm, and/or to refrain from transmitting any alarm-related signal to the rescuer device 108, based on the patient parameter(s) being outside of the alarm limit(s) 136, and/or based on the rescuer-related metric 142 being outside of the rescuer-related metric boundary(ies) 126. For instance, the alarm limit(s) 136 is utilized to control the medical device to refrain from outputting an alarm(s), and/or to refrain from transmitting any alarm-related signal(s) to the rescuer device 108, based on, and/or notwithstanding, the patient parameter(s) being above of a high limit of the alarm limit(s) 136. In such an instance or another instance, the alarm limit(s) 136 is utilized to control the medical device to output an alarm(s), and/or to transmit any alarm-related signal(s) to the rescuer device 108, based on, and/or notwithstanding, the patient parameter(s) being below a low limit of the alarm limit(s) 136, as illustrated in FIG. 1B.

Some or all of the management of the customized alarm limit(s) 136 may be the same as, or different from, some or all of the management of the customized alarm 134, as discussed above with reference to FIG. 1A. For example, such as with an instance in which the alarm limit(s) 136 have a wider range than the alarm limit(s) 132, the medical device 106 refrains from outputting any alarm, and/or from transmitting any alarm-related signal to the rescuer device 108, based on a patient heart rate of 40 bpm. For example, the medical device 106 identifies an alarm limit(s) 132, such as the high limit of the patient heart rate alarm, to be 135 bpm, and an alarm limit(s) 132, such as the low limit of the patient heart rate alarm, to be 35 bpm. The medical device 106, for example, determines and/or is set to utilize the wider range for the alarm limit(s) 136 based on being set and/or configured to decrease distractions for the rescuer 102 even further (e.g., a rescuer performing CPR and utilizing the limit(s) 136 may have a preference for a different alarm limit(s) than a rescuer performing CPR and utilizing the limit(s) 132). Alternatively or additionally, any other data is utilized to set and/or configure the alarm limit(s) 136 with the relatively wider range than the alarm limit(s) 132. For instance, the relatively wider range is utilized for circumstances in which there is a lot of background noise, and/or in such cases in which the patient has one or more other injuries to be treated, and/or being treated, by the rescuer 102. In such an instance or another instance, the relatively wider range is utilized for circumstances in which there are no other caregivers treating the patient 104, decreasing a likelihood that the alarm(s) will be easily silenced, such as in different circumstances with other caregivers being present.

In the example of FIG. 1B, the medical device 106 includes an input device(s) 138. In various cases, the input device(s) 138 receives treatment data 140 associated with the emergency scene 100A. For instance, the processor(s) 116 (e.g., via execution of the alarm customization module 120) determines a characteristic(s) of treatment by the rescuer 102 and to the patient 104. In some examples, the treatment data 140 includes the treatment characteristic(s), which is inferred from a treatment parameter(s) in the treatment data 140 and/or from other data (e.g., contextual data, as discussed below in further detail). For instance, the treatment characteristic(s) is inferred, such as in the sense that the treatment characteristic(s) of the patient 104 is deduced from a determination that the treatment parameter(s) in the treatment data 140 is related to a likely characteristic of the treatment by the rescuer 102 and to the patient 104.

The treatment data 140, for instance, is indicative of the treatment administered by the rescuer 102. By way of example, the treatment data 140 may be indicative of a context of the rescuer administer treatment, such as a rescuer movement(s) (e.g., acceleration(s)), a rescuer position(s), a rescuer utterance(s), a rescuer physiological parameter(s), a rescuer/treatment related sound(s), and so on, or any combination thereof. In some cases, the treatment data 140 indicates the chest compressions, based on the rescuer data 112, as discussed above with reference to FIGS. 1A, including motion artifact in the parameter(s) of the rescuer 102. The treatment data 140 is identified, in some cases, by identifying that the motion artifact temporally corresponds to the treatment received by the patient 104.

In some examples, the treatment characteristic(s) of the treatment by the rescuer 102 and to the patient 104 is related to a treatment of a medical condition of the patient 104. The treatment administered by the rescuer 102 is identified, for instance, by the medical device 106 and/or the rescuer device 108 identifying a role of the rescuer 102 at a rescue scene, which is the emergency scene 100B.

By way of example, the processor(s) 116 determines, by analyzing the treatment data 140 associated with the treatment and/or the emergency scene 100B, that the treatment includes chest compressions administered by the rescuer 102 and to the patient 104, likely experiencing a heart problem. Additionally or alternatively, the treatment, such as the chest compressions being administered, are determined by the processor(s) 116 analyzing contextual data associated with the emergency scene 100B. In alternative or additional examples, the contextual data is associated with a medical condition(s) of the patient 104. The treatment data 140, and possibly the contextual data, are utilized to determine that the treatment includes the chest compressions, such as part of CPR administered to the patient 104.

In some implementations, a set of alarms is managed in response to determining whether the patient 104 likely experiences the heart problem is identified in response to determining the treatment data 140, and, possibly the contextual data. For example, the processor(s) 116 (e.g., via execution of the alarm customization module 120) customizes the set of alarms, which are associated with the medical device 106, to obtain a customized set of alarms that is customized to the treatment data 140. In some instances, the customized set of alarms is customized to the characteristic of the treatment.

In various cases, the processor(s) 116 change a threshold(s) used for generating an alarm, such as a heart-related alarm (e.g., a pulse rate alarm, etc.). This is shown in FIG. 1B as the customized alarm limit(s) 136. That is, the low limit associated with the pulse rate alarm is changed from, for example, 135 bpm to 145 bpm, and/or the high limit associated with the pulse rate alarm may be changed from, for example, 45 bpm to 35 bpm. In other words, the limit(s) associated with the pulse rate alarm, for example, are from a narrower range to a wider range to decrease the sensitivity of the pulse rate alarm.

After obtaining the customized set of alarms, the processor(s) 116, in some instances, monitors a treatment parameter(s) of the treatment by the rescuer 102 to determine whether to generate an alarm(s) of the customized set of alarms. For example, the sensor 110 detects a treatment parameter(s) of the treatment by the rescuer 102, such as a chest compression parameter. In some cases, the processor(s) 116 receives the treatment parameter(s) via the sensor 110 and compares the treatment parameter(s) to a boundary(ies) (e.g., treatment boundary(ies)).

In various implementations, the medical device 106 automatically adjusts the alarm limit(s) 132 to be the customized alarm limit(s) 136. In response to determining the treatment parameter(s) exceeds the boundary(ies), and/or increases/graduates from being below the boundary(ies) to being above/beyond the boundary(ies), the alarm limit(s) 132, for example, are adjusted. In some instances, such as with a rescuer-related metric 142 (e.g., the compressions per minute) administered by the rescuer 102 increasing to be some value (e.g., 125 bpm) that is above a boundary (e.g., a rescuer-related metric boundary(ies) 126, as discussed above with reference to FIG. 1A, such as a high boundary of 120 compressions per minute), the alarm limit(s) 132 is adjusted to be the customized alarm limit(s) 136 with a relatively wider range. For example, the alarm limit(s) 132 is adjusted to be the customized alarm limit(s) 136, to decrease the sensitivity of the pulse rate alarm. For instance, the wider range is set to mitigate alarm fatigue of the rescuer 102. In other examples, the relatively wider range of the alarm limit(s) 136 with respect to the alarm limit(s) 132 is set, for example, due to the treatment parameter(s) exceeding a different high rescuer-related metric boundary, such as a boundary of 130 compressions per minute that is greater than the previous high rescuer-related metric boundary of 120 compressions per minute.

In alternative or additional examples, a customized alarm limit(s) set to a relatively narrower range with relatively greater sensitivity of the pulse rate alarm is changed to the customized alarm limit 136 with a relatively wider range for various reasons. For instance, the narrower range is changed to the wider range to enable the rescuer 102 to adjust treatment without distraction and, thereby, to perhaps enable the rescuer 102 to avoid receiving unhelpful and/or unnecessary feedback, so that the rescuer 102 is able to concentrate and/or perform more accurate chest compressions. The rescuer 102 performing relatively more accurate chest compressions, for example, also results in a decreased need for the alarm to be triggered. Whether increasing or decreasing the alarm sensitivity is more likely to result in decreasing alarm fatigue is determined based on various factors, including any of the treatment data 140 and/or the contextual data, and/or historical data related to other treatment data and/or contextual data related to previous emergency scenarios. Increasing or decreasing the alarm sensitivity is controlled, for instance, based on analysis by the AI model and/or user input.

In some cases, the alarm(s) and/or the alarm limit(s) 136 are controlled in various ways with respect to one another. For example, the alarm(s) and/or the alarm limit(s) 136 are controlled independently of one another. Alternatively, the alarm(s) are adjusted based on determining to not adjust the alarm limit(s) 136; and/or the alarm limit(s) are adjusted based on determining to not adjust the alarm(s). Adjusting the alarm(s) includes adjusting a volume, a pressure, a repetition, an escalation, a de-escalation, a trigger, a cut-off, etc., or any combination thereof, of the alarm(s) to be less onerous, distracting, unnecessary, redundant, and so on.

The treatment parameter(s) include an active status of chest compressions being performed and/or a number of how many of the chest compressions are performed per minute, for example. The treatment parameter(s), in some examples, are utilized by the medical device 106 and/or the rescuer device 108 to determine whether, and/or how, to display text indicating the alarm(s) (e.g., the advisory of the pulse-rate). In some examples, the rescuer metric(s), such as the rescuer-related metric 142, exceeding the rescuer-related metric boundary(ies) results in the medical device 106 and the rescuer device 108 refraining from displaying the customized alarm (e.g., refraining from outputting text indicating the advisory of the pulse-rate being less than 35 bpm) as a result of, and/or notwithstanding, the parameter(s) of the patient 104 exceeding the customized alarm limit(s) 136. By refraining from displaying the customized alarm, distractions to the rescuer 102 are minimized. In alternative examples, the rescuer metric(s) not exceeding the rescuer-related metric boundary(ies) results in the medical device 106 and/or the rescuer device 108 instructing the rescuer about the physiological parameter(s) of the patient 104 exceeding the customized alarm limit(s) 136 (e.g., such as for cases with the physiological parameter(s) of the patient 104 being above the high limit of the customized alarm limit(s) 136 or below the low limit of the customized alarm limit(s) 136). For instance, the rescuer metric(s) not exceeding the rescuer-related metric boundary(ies) results in the medical device 106 and/or the rescuer device 108 instructing the rescuer to perform chest compressions at a frequency, a depth, a location, or a duty cycle, based on the physiological parameter(s) of the patient 104 exceeding the customized alarm limit(s) 136. In response to determining the rescuer metric(s) do not exceed the rescuer-related metric boundary(ies), the processor(s) 116, for instance, generates an alarm(s) of the customized set of alarms based on the physiological parameter(s) of the patient 104 exceeding the customized alarm limit(s) 136.

In various types of implementations, various alarms of various types are output in response to determining the treatment metric(s) do not exceed the boundary(ies). For example, the medical device 106 outputs an alarm(s), which is in the form of a visual alarm (e.g., “Advisory: Pulse Rate <35) presented on a display of the medical device 106 and an audible alarm (e.g., a siren, a periodic “beep” sound, etc.) output via a speaker(s) of the medical device 106. Of course, the alarm may be output in other ways, such as a flashing LED(s), a vibration of a haptic actuator(s), and/or the like. The alarm is generated, for instance, in response to identifying the treatment data 140, and, possibly, identifying, via the rescuer data 112 and in the treatment data 140, the role of the rescuer.

By customizing the alarm(s) based on the treatment characteristic of the treatment administered by the rescuer 102, the alarm(s) is rendered more effective in alerting the rescuer 102 as to a current medical condition(s) (e.g., a respiratory problem) of the patient 104, of which the rescuer 102 may be unaware. For example, the medical device 106 utilizing the treatment drive alarm(s) reduces or eliminates alarms that are onerous, distracting, unnecessary, redundant, and so on.

The contextual data associated with the emergency scene 100B and/or a medical condition(s) of the patient 104 include, for instance, a physiological parameter(s) of the patient 104, a context of the emergency scene 100A, a location of the medical device 106, audio at the emergency scene 100A, images and/or video of the emergency scene 100A, environmental conditions (e.g., heatwaves, air quality alerts, flooding alerts, etc.), and/or the like. Accordingly, the input device(s) 138 that is configured to receive the contextual data take one or more of many forms, such as a microphone(s), a camera(s), a touch-sensitive display(s), a keypad(s), a cursor control device(s), a location-determining device(s) (e.g., a Global Positioning System (GPS) receiver), a transceiver(s), or any combination thereof. The output device(s) 124 includes at least one of a display(s), a light emitting element(s) (e.g., a light emitting diode(s) (LED(s))), an electrochromic material(s), a speaker(s), a haptic actuator(s), a transceiver(s), a printer(s), or any combination thereof. The examples described herein, the output device(s) 124 is configured to output, and/or to not output, any of the alarm(s).

In various implementations, the treatment data 140 includes, alternatively or additionally to chest compression related data, other type of medical treatment data. For instance, the treatment data 140 includes any type of treatment parameter(s) of the rescuer 102 related to any type of treatment(s) (e.g., chest compressions). In some cases, the treatment parameter(s) include a blood pressure, a respiration rate, a temperature, an acceleration, a location, a vocal sound, a pupil movement, and so on, or any combination thereof.

In various implementations, a type of the alarm(s) includes a CPR-based alarm type corresponding to the rescuer-related treatment (e.g., the chest compressions). In some cases, the alarm instructs the rescuer 102 to change a frequency, a depth, a location, or a duty cycle of the chest compressions.

In various implementations, the alarm for adjusting the compressions, such as any of a characteristic(s) (e.g., a frequency, a depth, a location, or a duty cycle), is output notwithstanding a rescuer-related metric(s) exceeding a boundary(es) 126. By outputting certain types of alarm(s) related to, for example, problems with, and/or suggestions for, treatment currently being administered at a less than ideal level and/or way, the rescuer 102 is able to improve the treatment. In some cases, outputting of the alarm(s) related to, for example, problems with, and/or suggestions for, treatment currently being administered at a less than ideal level and/or way may be performed based on the rescuer-related metric(s) (e.g., rescuer-related metric(s) 142) exceeding the boundary(es) 126, such as based on the medical device 106 identifying that the rescuer-related metric(s) exceed the boundary(es) 126 and/or that the treatment is currently being administered at a less than ideal level and/or way. Whether to not output (e.g., or to output) certain types of alarm(s) related to patient parameter(s) and/or whether to output (e.g., or to not output) certain types of alarm(s) related to rescuer and/or treatment parameter(s) is, in some instances, determined by the medical device 106 (e.g., such as via analysis by the AI model(s)).

The treatment includes various types of treatment. Alternatively or additionally to chest compressions, the treatment includes, by way of example, assisted ventilation, administration of a medication, etc. A type of the alarm(s) includes, for example, a ventilation-based alarm type corresponding to the assisted ventilation. In various cases, the alarm instructs the rescuer 102 to change a rate of the assisted ventilation, to change a tidal volume of the assisted ventilation, to administer a medication, or to check for a leak between a ventilation device and an airway of the patient.

The customized alarm includes various types of alarms. The customized alarm includes instructions, by way of example, to change a rate of the assisted ventilation, to change a tidal volume of the assisted ventilation, to administer a medication, to check for a leak between a ventilation device and an airway of the patient, and so on, or any combination thereof.

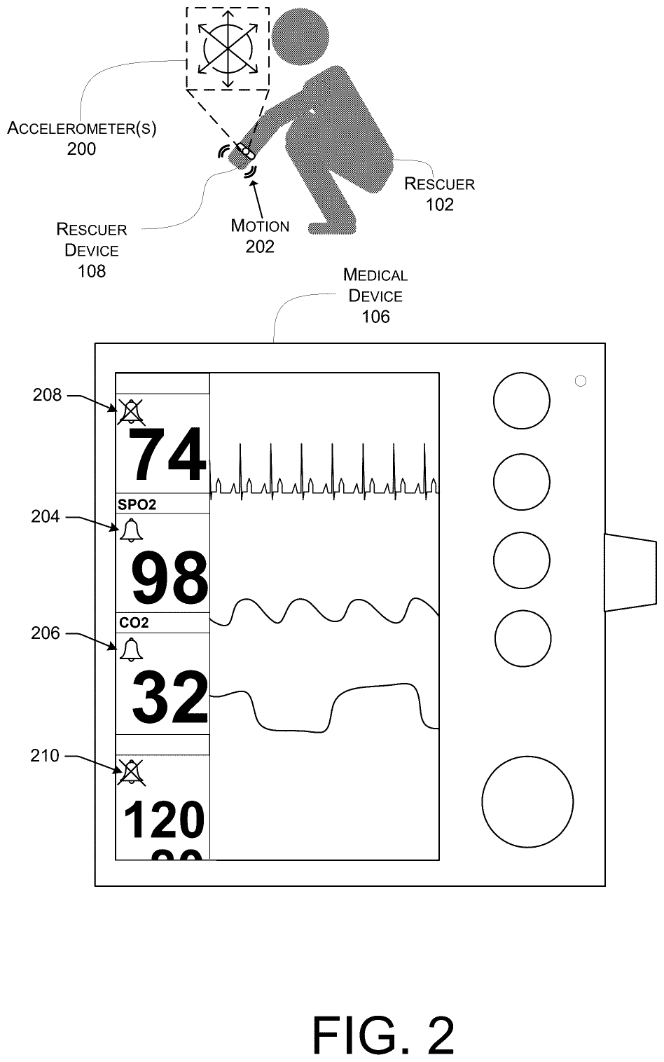

FIG. 2 illustrates a particular example of a rescuer device configured to determine that a rescuer is likely performing treatment to a patient, and to customize a set of alarms of the medical device to obtain a customized set of alarms tailored to the determined treatment. For example, the rescuer device, such as the rescuer device 108, as discussed above with reference to FIGS. 1A and 1B, processes movement data representing a motion by the rescuer 102 in an environment of the medical device 106 and the rescuer device 108. In various instances, the medical device 106 and/or the rescuer device 108 customizes a set of alarms to obtain a customized set of alarms tailored to the treatment by the, such as the rescuer 102, as discussed above with reference to FIGS. 1A and 1B. The sensor 110 of the rescuer device 108 includes input device(s) in a form of accelerometer(s) 200, in some instances. In some cases, the accelerometer(s) 200 includes a movement sensor(s). The accelerometer(s) 200, for example, is utilized to detect motion 202 associated with the rescuer 102.

Alternatively or additionally, such as in the example of FIG. 2, the rescuer device, such as the rescuer device 108, as discussed above with reference to FIGS. 1A and 1B, includes the input device(s) in the form of a meter(s), such as the accelerometer(s) 200, a heart rate monitor(s), etc., or any combination thereof. For instance, rescuer data, such as the rescuer data 112, as discussed above with reference to FIGS. 1A and 1B, is utilized to generate the treatment data 140 received by an input device (e.g., the input device(s) 138, as discussed above with reference to FIG. 1B) of the medical device 106. In various cases, the medical device 106 and the rescuer device 108 exchange signals utilized to manage the rescuer data 112, the treatment data 140, any data utilized to manage a rescuer-related metric boundary(ies), any data utilized to manage a patient related alarm(s) and/or an alarm limit(s), etc., or any combination thereof.

In some embodiments, the device(s) (e.g., the medical device 106 and/or the rescuer device 108) are utilized to capture various types of data. For example, the meter(s) of the rescuer device 108 captures a movement parameter(s) (e.g., of a movement), a heart rate, etc., of the rescuer 102 in an environment, and so on, or any combination thereof, of the rescuer device 108 while the rescuer device 108 is worn by the rescuer 102. In such an example or another example, the medical device 106, such as the processor(s) 116 (e.g., via execution of the alarm customization module 120), and/or the rescuer device 108, process the treatment data 140 to determine a recognition result (e.g., a movement recognition result, a heart rate recognition result, etc., or any combination thereof), and determine a characteristic(s) of the rescuer 102 by analyzing the recognition result. Alternatively or additionally, individual ones of the rescuer device 108 and the medical device 106 includes an input device in the form of a video camera capturing video data utilized to determine a recognition result. The video data may be utilized to determine a characteristic(s) of the rescuer 102 indicative of CPR chest compressions.

In various examples, the medical device 106, such as the processor(s) 116 (e.g., via execution of the alarm customization module 120), and/or the rescuer device 108, customize a set of alarms associated with the medical device 106 and/or the rescuer device 108 to obtain a customized set of alarms that is customized based on the characteristic of the rescuer 102. The set of alarms, which is associated with parameter(s) of the patient 104, includes, for instance, a respiratory alarm 204, a carbon dioxide alarm 206, a heart rate alarm 208, and an arterial pressure alarm 210.

The alarm(s) are managed, for example, based on rescuer data 112 and/or the treatment data 140, in various ways. In some implementations, the processor(s) 116 may deprioritize or mute output of a heart rate-related alarm(s), such as the heart rate alarm 208 and/or the arterial pressure alarm 210, associated with the patient 104 under an additional alarm(s) of the set of alarms. Additionally, or alternatively, the processor(s) 116 may enable or activate the additional alarm(s) of the set of alarms that are not associated with the medical condition (e.g., heart problem). Additionally, or alternatively, the processor(s) 116 may enable or activate the additional alarm(s) of the set of alarms that related to treatment of other medical conditions.

For example, FIG. 2 shows that a first additional alarm and a second additional alarm have been enabled or activated. The first additional alarm might be the respiratory alarm 204, for example, which may not be considered to be associated with a condition(s) other than a heart medical condition, or which may not be related to treatment for the other condition(s). The second additional alarm might be the carbon dioxide alarm 206, for example, which may not be considered to be relatively more indicative of information utilized to determine whether to perform other treatment(s). Thus, enabling or activating the alarms 204 and/or 206 may allow the rescuer 102 to focus on the alarms 204 and/or 206 that have been prioritized over the alarms 208 and/or 210, to avoid distracting alarms related to CPR treatment being provided to the rescuer 102.

After obtaining the customized set of alarms, the processor(s) 116, for example, is configured to monitor a parameter(s) of the rescuer 102 to determine whether to generate an alarm(s) of the customized set of alarms. For example, the sensor 110 detects a parameter(s) of the rescuer 102, such as a movement (or “motion”) parameter(s), a heart rate, and so on, or any combination thereof. The processor(s) 116 may receive the rescuer-related parameter(s) via the sensor 110. In some instances, the processor(s) 116, upon receiving the rescuer-related parameter(s), compares the rescuer-related parameter(s) to a boundary(ies). In response to comparing the rescuer-related parameter(s) to the boundary(ies), the processor(s) 116, for example, generate an alarm(s) of the customized set of alarms based on a parameter of the patient 104 exceeding an alarm limit(s). In various instances, if the movement (e.g., movement corresponding to a number of chest compressions per unit of time), the heart rate, and so on, or any combination thereof is above a boundary(ies) (e.g., a movement metric boundary corresponding a number of chest compressions per unit of time threshold, and so on, or any combination thereof), the processor(s) 116 may determine that this rescuer-related parameter is greater than a boundary, and the processor(s) 116 may customize the heart rate alarm 208 and/or the arterial pressure alarm 210 in this scenario. The processor(s) 116, for instance, causes the alarm(s) to be output via the output device(s) 124 at a relatively lower volume and/or occurrence level, as described herein. By cause the alarm(s) to be output via at the relatively lower volume and/or occurrence level, alarm fatigue of the rescuer 102 may be reduced and/or mitigated. In various instances, management of an alarm(s) and/or an alarm limit(s) associated with the rescuer device 108 is performed in a similar way as, or a different way from, management of the alarms 204-210, and/or the alarm limit(s), associated with the medical device 106.

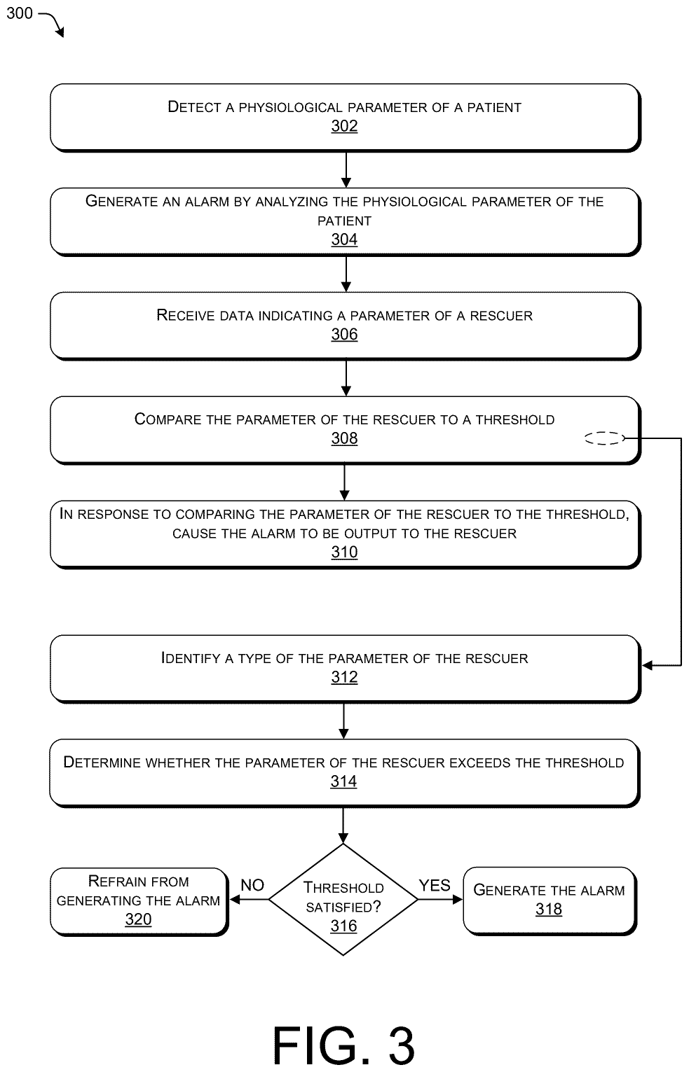

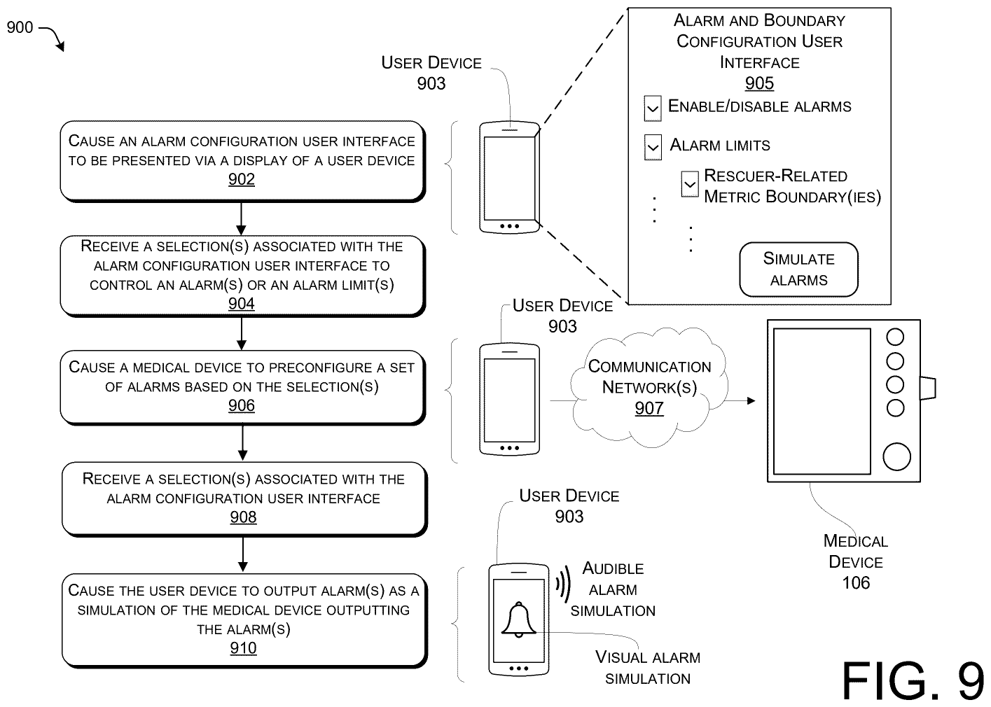

FIGS. 3-9 illustrate processes performed by one or more systems, devices, or entities described herein. For example, the processes illustrated in FIGS. 3-8 may be implemented by the medical device 106, or at least one processor, such as the processor(s) 116, configured to execute instructions. As another example, the process illustrated in FIG. 9 may be implemented by a user device, or at least one processor configured to execute instructions. The processes described herein represent sequences of operations that can be implemented in hardware, software, or a combination thereof. In the context of software, the blocks represent computer-executable instructions stored on one or more computer-readable storage media that, when executed by a processor(s), perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be combined in any order and/or in parallel to implement the processes. In some examples, an operation(s) of the process may be omitted entirely. Moreover, the processes described herein can be combined in whole or in part with each other or with other processes.

FIG. 3 illustrates an example process 300 for determining a parameter of a rescuer from context of an emergency scene where the rescuer and a patient are located, and customizing a set of alarms of a medical device to the parameter of the rescuer in order to output an alarm that is customized to the rescuer. At 302, for instance, a medical device (e.g., the medical device 106) (e.g., and/or a patient device) detects a physiological parameter of a patient 104. In some instances, the medical device 106 includes, and/or be coupled to, a sensor(s) utilized to receive patient data. In various cases, the medical device 106 receives contextual data associated with the emergency scene 100B, such as data associated with the patient 104. The medical device 106, for instance, detects a physiological parameter(s) via the patient data, such as in response to receiving the patient data with the physiological parameter(s) and/or receiving the patient data from which the physiological parameter(s) is obtained/extracted.

In some examples, a transceiver(s) 122 of the medical device 106 is configured to receive the rescuer data 112. For instance, the rescuer data 112 is received from the rescuer device 108 based on an input device(s) of the rescuer device 108 identifying data utilizing, for instance, an accelerometer, a heart rate monitor, a microphone(s), a camera(s), a touch-sensitive display(s), a keypad(s), a cursor control device(s), a location-determining device(s) (e.g., a GPS receiver), a transceiver(s), or any combination thereof. In various examples, an input device(s) 138 of the medical device 106 is possibly configured to identify the treatment data 140, which is indicative of the treatment administered by a rescuer 102 and generated via the medical device 106 analyzing a parameter(s) of the rescuer 102, and/or a treatment administered by the rescuer 102 at the emergency scene 100A. The parameter(s) of the rescuer 102 is identified utilizing the rescuer device 108, for example. The context includes, for example, location data, audio data, image data (e.g., still images, video, etc.), environmental data, or any combination thereof.

At 304, for instance, the medical device 106 generates an alarm by analyzing the physiological parameter of the patient 104. A physiological parameter(s) of the patient 104 is included in, and/or utilized to determine, the contextual data. In some examples, the processor(s) 116 of the medical device 106 is configured to determine a characteristic(s) of the patient 104 by analyzing the physiological parameter(s) and/or the contextual data at 304. The medical device, for instance, generates an alarm(s), by executing the alarm customization module 120 and/or by processing the contextual data to identify characteristic(s) of the patient 104 in the contextual data. In alternative or additional examples, the contextual data is utilized to determine the characteristic(s) of the patient 104.

In some implementations, the medical device 106 compares the physiological parameter(s) of the patient 104 to the threshold. For example, comparing the physiological parameter(s) of the patient 104 to the threshold includes determining that treatment (e.g., assisted ventilation) administered to the patient 104 is ineffective. In some cases, the alarm includes an advisory that the patient parameter(s) exceeds a limit(s), an instruction to change numbers of compressions per minute, an instruction to check for a leak between a ventilation device and a face of the patient 104, an instruction to change a ventilation parameter of the assisted ventilation, etc., or any combination thereof.

In various cases, the medical device 106 is included in a system with a transceiver that receives a communication signal indicating the heart rate of the rescuer 102, and/or transmits a communication signal indicating an alarm. The communication signal indicating the heart rate of the rescuer 102 is received from the rescuer device 108, such as from a transceiver (or “first transceiver”) of the rescuer device 108. The communication signal indicating the alarm is transmitted to the rescuer device 108. In various cases, the medical device 106 includes a measurement circuit that detects an electrocardiogram (ECG) of a patient. In some instance, the medical device 106 includes a transceiver (or “second transceiver”) that receives the communication signal(s) indicating the heart rate of the rescuer 102. In those or other examples, the medical device 106 determines that the ECG is indicative of an arrhythmia. In those or other examples, the medical device 106 (e.g., the processor(s) 116), in response to determining that the ECG is indicative of the arrhythmia, generate the alarm(s).

The communication signals are transmitted in various ways. For example, a communication signal(s) associated with the parameter(s) (e.g., the blood pressure, the respiration rate, the temperature, the acceleration, the location, the vocal sound, the pupil movement, the pressure on the rescuer hand, etc., or any combination thereof) of the rescuer 102 includes a short-rage communication-based proximity signal transmitted to the medical device 106 (e.g., a treatment device) and from the rescuer device 108. In such an example or another example, a communication signal(s) associated with the alarm(s) includes a short-rage communication-based proximity signal transmitted by the medical device 106 and to the rescuer device 108.

In various implementations, the alarm at 304 is generated by the trained AI model(s). In some examples, the alarm at 304 is generated by analyzing the treatment data 140 and/or the contextual data, such as by providing the treatment data 140 and/or the contextual data as input to the trained AI model(s) (e.g., a machine learning (ML) model(s)) and receiving the characteristic(s) of the rescuer 102 and/or the patient 104 as output from the trained AI model(s). In some examples, generating the alarm(s) at 304 includes accessing, from a datastore, historical data associated with physiological parameter(s) and/or characteristics of rescuers and/or patients located at emergency scenes in the past and analyzing a subset of the historical data based on contextual data to determine the physiological parameter(s) and/or the characteristic(s) of the rescuer 102 and/or the patient 104.

In various examples, the trained AI model(s) are implemented in the medical device 106 and/or another device(s). The other device(s) includes, for instance, an external device(s), such as in a cloud computing network. In various examples, the other device(s) are communicatively coupled to the medical device 106 and/or the rescuer device 108.

In various implementations, the characteristic(s) of the patient 104 is identified in various ways, such as based on the physiological parameter(s) of the patient 104. In some examples, the characteristic(s) of the patient 104 is inferred from the contextual data in the sense that the characteristic(s) of the patient 104 is deduced from the contextual data as a likely characteristic of the patient 104. In some examples, the characteristic(s) of the patient 104 is a medical condition of the patient 104, such as a respiratory problem (e.g., asphyxiation, shortness of breath, etc.), a cardiovascular problem (e.g., a heart attack), and/or the like. In some examples, the characteristic(s) of the patient 104 is an attribute(s) or a classification(s) of the patient 104 as one of multiple class labels (e.g., obese or not obese, adult or child, conscious or unconscious, breathing or not breathing, etc.), which can be used to infer a medical condition of the patient 104.

At 306, for instance, the medical device 106, receives data indicating a parameter of a rescuer 102. For instance, data indicating the parameter is received from the rescuer device 108. In some examples, the data includes the rescuer data 112. The rescuer data 112 can be analyzed, for example by the medical device 106, and/or an additional device(s) in communication with the medical device 106. In some implementations, the rescuer data 112 is analyzed by providing the rescuer data 112 as input to a trained AI model(s) (e.g., an ML model(s)). In some cases, the medical device 106 identifies, and/or receives as output from the trained AI model(s), a characteristic(s) (e.g., a role(s)) of the rescuer 102. In some examples, receiving the characteristic(s) includes accessing, from a datastore, historical data associated with parameters and/or characteristics of rescuers located at emergency scenes in the past and analyzing a subset of the historical data based on the parameters and/or the characteristics to determine the parameter(s) of the rescuer 102. In some examples, the characteristic(s) is inferred from the rescuer data 112 in the sense that the characteristic(s) of the rescuer 102 is deduced from the rescuer data 112 to identify a likely characteristic of the rescuer 102. In some examples, the characteristic(s) of the rescuer 102 includes a type of treatment administered by the rescuer 102 to the patient 104, such as chest compressions, assisted ventilation and the like, or any combination thereof.

In some examples, the characteristic(s) output from the trained AI model(s) is utilized, based on the characteristic(s) being previously, initially, and/or currently unascertainable, unknown, unidentified, unobtainable, inaccessible, etc., or any combination thereof. In alternative examples, the characteristic(s) of the rescuer 102 output from the trained AI model(s) is utilized based on the characteristic(s) being previously, initially, and/or currently not readily ascertainable, known, identifiable, obtainable, accessible. In alternative or additional examples, the characteristic(s) of the rescuer 102 output from the trained AI model(s) is utilized based on the characteristic(s) being difficult (e.g., slightly difficult or severely difficult) and/or time consuming to ascertain, know, identify, obtain, access, etc., or any combination thereof.

In some examples, the processor(s) 116 of the medical device 106 is configured to customize a set of alarms at 306 by executing the alarm customization module 120 and/or by using the parameter(s) and/or the characteristic(s) of the rescuer 102 to look up information on how to customize the set of alarms to the parameter(s) and/or the characteristic(s) of the rescuer 102. In general, the customized set of alarms obtained at 306 are tailored to the rescuer 102, the rescuer data 112, etc., or any combination thereof. Alternatively or additionally, the customized set of alarms obtained at 306 are tailored to the treatment data 140. To illustrate with one example, customizing the set of alarms at 306 may include changing a threshold(s) used for generating an alarm(s) (e.g., changing the high and/or low limits associated with a particular alarm(s) to make the alarm(s) more or less sensitive and/or more appropriately tailored to the rescuer 102, the rescuer data 112, the treatment data 140, etc., or any combination thereof.

At 308, for instance, the medical device 106 compares the parameter of the rescuer 102 to a threshold (e.g., a boundary). By way of example, the medical device 106 compares the parameter(s), which includes various type(s) of parameter(s) (e.g., a heart rate, a movement parameter(s) such as for chest compressions per unit of time, and so on, or any combination thereof) to a threshold (boundary), which includes various type(s) of threshold(s) (e.g., boundary(ies)) (e.g., a heart rate threshold, a movement parameter threshold(s), and so on, or any combination thereof). In various examples, a parameter(s) being compared a threshold(s) includes a rescuer parameter(s), another parameter(s), and so on, or any combination thereof. In various implementations, the parameter(s) being compared the threshold(s) possibly includes a treatment parameter(s). In some examples, the movement parameter(s) include a type, amount, level, severity, rapidity, repetitive, and so on, or any combination thereof, of the movement. In various implementations, the movement parameter(s) include a number, a frequency, a depth, a location, or a duty cycle, and so on, or any combination thereof, of chest compressions. In some instances, the threshold(s) includes a rescuer parameter threshold(s), another threshold(s), or any combination thereof. In various cases, the threshold(s) possibly includes a treatment parameter threshold(s).

At 310, for instance, the medical device 106, in response to comparing the parameter of the rescuer 102 to the threshold, causes the alarm to be output to the rescuer 102. The medical device 106 determines whether to generate an alarm(s) of the customized set of alarms by analyzing the parameter(s) of the rescuer 102, and possibly the parameter(s) of the treatment. In some examples, the processor(s) 116 of the medical device 106 is configured to cause the alarm(s) to be output at 308 by processing the parameter(s) of the rescuer 102 or otherwise using the parameter(s) to determine whether to cause the alarm(s) to be output or refrain from causing the alarm(s) to be output. In some examples, the alarm(s) caused to be output at 308 is associated with the rescuer 102, and/or the treatment of the rescuer 102, and/or with a medical condition of the patient 104 determined at 304.

At 312, for instance, to compare the parameter of the rescuer 102 to a threshold, the medical device 106 identifies a type of the parameter of the rescuer 102. In some examples, the type of the parameter of the rescuer 102 is identified, in response to receiving data (e.g., data identified using a sensor(s) 110) indicating the parameter(s) of the rescuer 102. In various cases, the data being received includes the rescuer data 112.

At 314, for instance, the medical device 106 determines whether the parameter of the rescuer 102 exceeds the threshold. By way of example, the medical device 106 determines whether a parameter(s) (e.g., a movement parameter(s), a heart rate, and so on, or any combination thereof) exceeds a threshold(s) (e.g., a boundary(ies)) (e.g., a movement parameter threshold(s), a heart rate threshold, and so on, or any combination thereof).

At 316, for instance, the medical device 106 determines the threshold is satisfied and proceeds to step 318. Alternatively, for instance, the medical device 106 determines the threshold is not satisfied and proceeds to step 320.

At 318, for instance, the medical device 106 generates the alarm. The alarm is generated, in some examples, in response to the medical device 106 determining the parameter(s) (e.g., the movement parameter(s), the heart rate, and so on, or any combination thereof) does not exceed the threshold(s) (e.g., the movement parameter threshold(s), the heart rate threshold, and so on, or any combination thereof).

In some examples, the medical device 106 determines that the heart rate of the rescuer 102 is below a threshold. In those or other examples, the medical device 106, response to determining that the heart rate of the rescuer is below the threshold, causes the transceiver (e.g., the second transceiver) to transmit the communication signal indicating the alarm.

At 320, for instance, the medical device 106 refrains from generating the alarm. The alarm is not generated, in some examples, in response to the medical device 106 determining the parameter(s) (e.g., the movement parameter(s), the heart rate, and so on, or any combination thereof) exceeds the parameter(s) (e.g., the movement parameter(s), the heart rate, and so on, or any combination thereof) does not exceed the threshold(s) (e.g., the heart rate threshold, the movement parameter threshold(s), and so on, or any combination thereof). By not generating the alarm, alarm fatigue of the rescuer 102 may be reduced and/or mitigated. By utilizing the movement and/or the heart rate of the rescuer 102 to determine whether to generate the alarm, fatigue associated with the rescuer 102 undergoing high levels of exertion may be reduced and/or mitigated.

It is to be appreciated that other techniques for causing the alarm(s) to be output at 310 may be implemented, in some examples. For example, causing the alarm(s) to be output at 310 may include providing the parameter(s) of the rescuer 102 as input to a trained AI model(s) (e.g., an ML model(s)) and receiving a classification (e.g., alarm to be triggered or alarm not to be triggered) as output from the trained AI model(s).

In some examples, a training dataset(s) is used to train the ML model(s). For instance, the training dataset(s) includes features and labels. However, the training dataset(s) may be unlabeled, in some examples. Accordingly, the ML model(s) are trained, in various cases, using any suitable learning technique, such as supervised learning, unsupervised learning, semi-supervised learning, reinforcement learning, and so on. The features included in the training dataset(s) are represented, for instance, by a set of features, such as in the form of an n-dimensional feature vector of quantifiable information about an attribute of the training dataset(s). The training dataset(s) include any of the various types of data/information input to the ML model(s), and/or any other, previous, subsequent, contemporaneous data/information of a similar, and/or a different, type.

In some embodiments, the ML model(s) represent a single model or an ensemble of base-level ML models, and are implemented as any of various types of ML models. For example, suitable ML models for use by the techniques and systems described herein include, without limitation, neural networks (e.g., generative adversarial networks (GANs), deep neural networks (DNNs), recurrent neural networks (RNNs), etc.), tree-based models (e.g., extreme Gradient Boosting (XGBoost) models), support vector machines (SVMs), kernel methods, random forests, splines (e.g., multivariate adaptive regression splines), hidden Markov model (HMMs), Kalman filters (or enhanced Kalman filters), Bayesian networks (or Bayesian belief networks), multilayer perceptrons (MLPs), expectation maximization, genetic algorithms, linear regression algorithms, nonlinear regression algorithms, logistic regression-based classification models, or an ensemble thereof. An “ensemble” can comprise a collection of ML models whose outputs (predictions) are combined, such as by using weighted averaging or voting. The individual ML models of an ensemble can differ in their expertise, and the ensemble can operate as a committee of individual ML models that is collectively “smarter” than any individual ML model of the ensemble

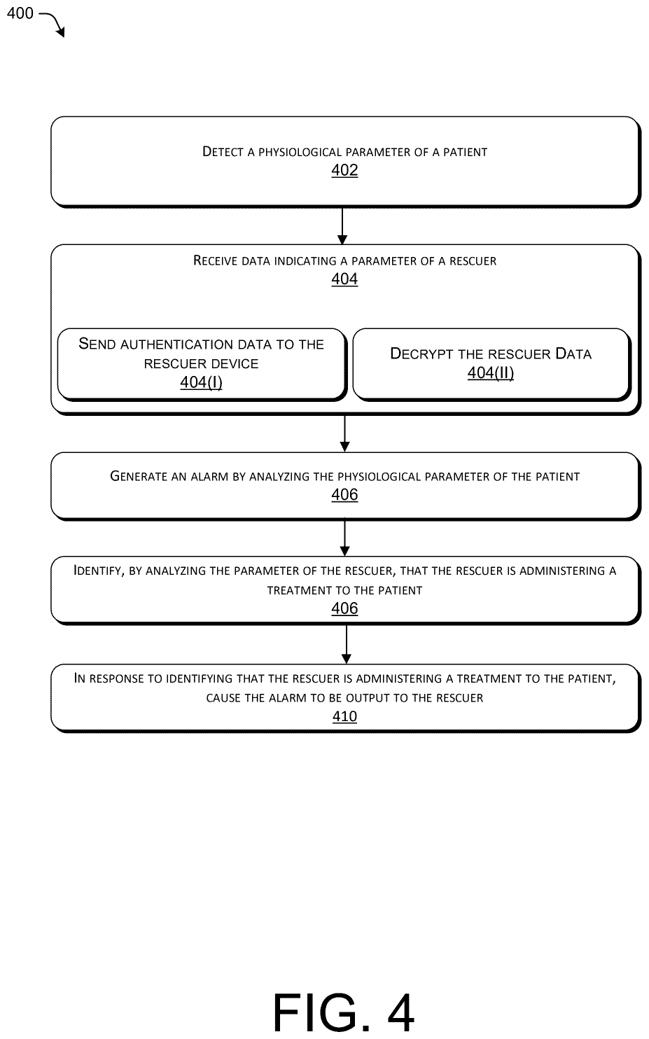

FIG. 4 illustrates an example process for determining, from treatment data determined by a medical device, a parameter of a rescuer to which a type of treatment administered by the rescuer corresponds, and customizing a set of alarms of the medical device to the type of treatment to output an alarm that is customized to the type of treatment. In various cases, the type of treatment is customized to the rescuer 102, such as to the parameter(s) (e.g., the parameter(s) utilized to identify the role(s)) of the rescuer 102.

At 402, for instance, a medical device detects a physiological parameter of a patient. In some examples, a physiological parameter(s) of the patient (e.g., a patient 104) is detected by the medical device (e.g., a medical device 106), such as by a sensor(s) of the medical device 106 and/or by a sensor(s) communicatively coupled to the medical device 106. In some examples, the medical device 106 identifies the physiological parameter(s) in patient data, which is generated by the sensor(s) and associated with the patient 104.

At 404, for instance, the medical device 106 receives data indicating a parameter of a rescuer. In some examples, the parameter of the rescuer (e.g., the rescuer 102) is received in rescuer data 112, such as from a rescuer device 108, which possibly includes a sensor 110. In those or other examples, a parameter(s) of the rescuer 102 includes a heart rate, a blood pressure, a respiration rate, a temperature, an acceleration, a movement (e.g., movement corresponding to a number of chest compressions per unit of time), a location, a vocal sound, a pupil movement, and so on, or any combination thereof,

At 404(I), for instance, the medical device 106 sends authentication data to the rescuer device 108. In some examples, the authentication data is sent in response to receiving the data indicating the parameter of the rescuer 102.

At 404(II), for instance, the medical device 106 decrypts the rescuer data 112. In some examples, the rescuer data 112 is decrypted in response to sending the authentication data.

At 406, for instance, the medical device 106 generates an alarm by analyzing the parameter(s) of the rescuer 102 and/or the physiological parameter of the patient 104. In some examples, a physiological parameter(s) of the patient 104 is determined as being indicative of a medical condition(s) of the patient 104 for which an alarm(s) is to be generated.

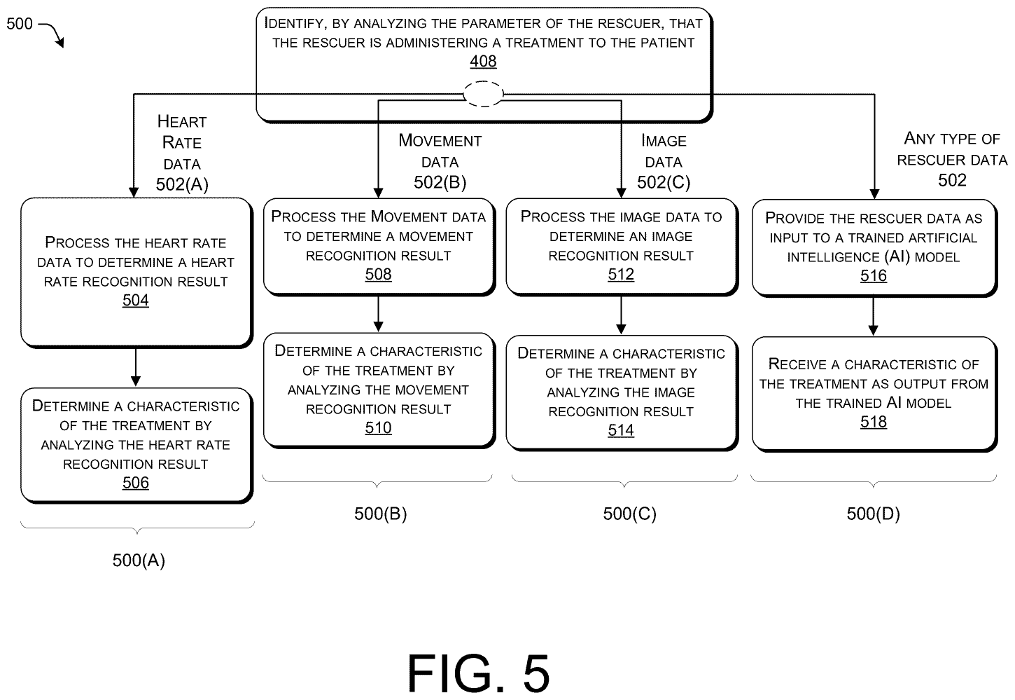

At 408, for instance, the medical device 106 identifies, by analyzing the parameter of the rescuer 102, that the rescuer is administering a treatment to the patient 104. The parameter of the rescuer 102 received by a transceiver(s) 122 and in the rescuer data 112 is analyzed to identify a characteristic(s) of the treatment by the rescuer 102. An example technique for identifying that the rescuer is administering a treatment to the patient 104 at 408 will be discussed below with respect to sub-blocks 500A-500D of FIG. 5.

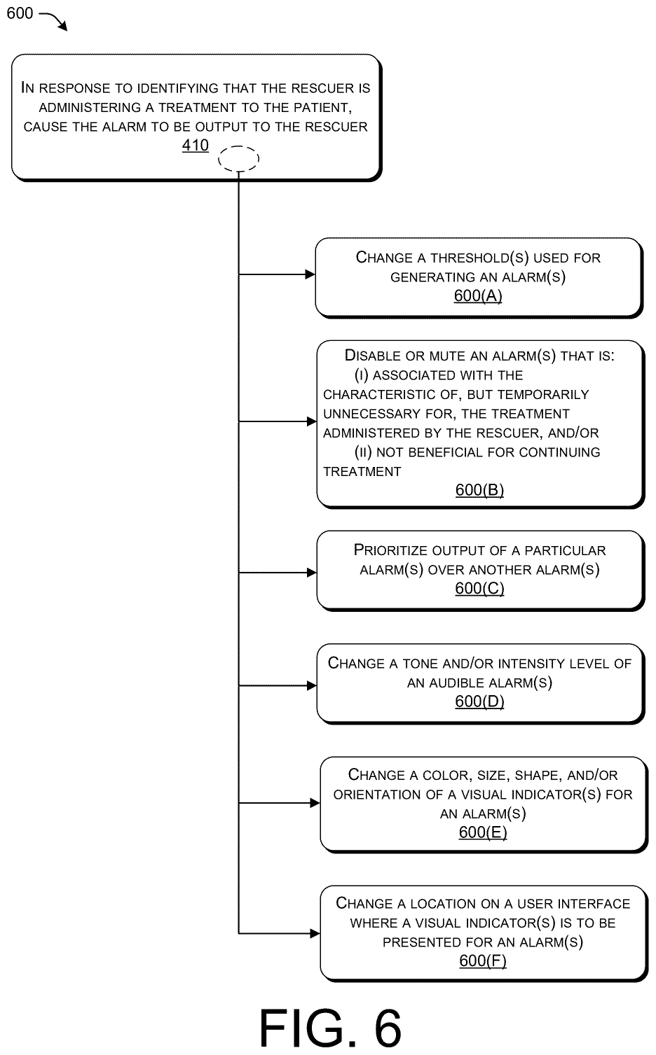

At 410, for instance, the medical device 106, in response to identifying that the rescuer 102 is administering a treatment to the patient 104, causes the alarm to be output to the rescuer 102. The alarm(s) is generated and output to the rescuer 102 in response to identifying the characteristic(s) of the treatment by the rescuer 102. The alarm(s) is output from among a customized set of alarms in response to customizing the set of alarm(s) to the characteristic(s) of the treatment by the rescuer 102, to reduce alarm fatigue of the rescuer 102. An example technique for causing the alarm(s) to be output at 410 will be discussed below with respect to sub-blocks 600A-600F of FIG. 6. In various cases, the alarm(s) is output from among the customized set of alarms in response to customizing the set of alarm(s) to the characteristic(s) (e.g., role) of the rescuer 102.

FIG. 5 illustrates example techniques in a process 500 for determining a characteristic(s) of a treatment of a rescuer from rescuer data, and context of an emergency scene where the rescuer is located. As indicated in FIG. 5, the example techniques illustrated in FIG. 5 may be performed as sub-operations of block 408 of the process 400 described above. With reference to a first technique 500(A), the rescuer data 112 includes heart rate data 502(A) representing a heart rate(s) of a rescuer 102 in an environment of the medical device 106 while the medical device 106 is located at the emergency scene 100B. For example, the rescuer device 108 includes a sensor 110 in the form of a heart rate monitor(s).

At 504, for instance, the medical device 106 processes the heart rate data to determine a heart rate recognition result. In some examples, the rescuer data 112 includes the heart rate data, which is received by the input device 138. In this example, the heart rate monitor(s) may capture a heart rate of the rescuer 102 while the rescuer 102 and the medical device 106 is located at an emergency scene 100B.

In various cases, the processor(s) 116 of the medical device 106 processes heart rate data 502(B) to determine a heart rate recognition result(s). Any suitable technology can be used to process the heart rate data 502(B) to determine the heart rate recognition result. In some examples, event detection software can recognize heart rate patterns (e.g., values, frequencies, etc.).

At 506, for instance, the processor(s) 116 of the medical device 106 determines a characteristic of the treatment by analyzing the heart rate recognition result. In various examples, the processor(s) 116 determines the treatment data 140, which includes a characteristic(s) of the treatment by the rescuer 102, by analyzing the heart rate recognition result. For example, if a heart rate recognition result is indicative of a heart rate above 105 bpm, the processor(s) 116 may determine, from the heart rate recognition result(s), that the characteristic of the treatment is likely cardiac massage(s). In another example, the rescuer 102 may have the heart rate rise above 165 bpm, and the processor(s) 116 may determine, from a heart rate recognition result indicative of the heart rate rising above 160 bpm, that the characteristic of the treatment more likely includes chest compressions than cardiac massage(s). The parameter(s) of the rescuer 102, such as the heart rate, is utilized to identify a characteristic (e.g., a role) of the rescuer 102, in some cases. A characteristic(s) (e.g., a role(s)) of the rescuer 102, for instance, is identified.

With reference to a second technique 500(B), the rescuer data 112 includes movement data 502(B) representing movement of a rescuer 102 in an environment of the medical device 106 while the medical device 106 is located at the emergency scene 100B. For example, the medical device 106 may include an input device(s) 138 in the form of an accelerometer(s) 200. At 508, for instance, the medical device 106 processes the movement data 502(B) to determine a movement recognition result. In some examples, the rescuer data 112 includes the recognition data, which is received by the input device(s) 138. In various cases, the recognition data includes data in the form of movement data 502(B). In this example, the accelerometer(s) 200 may capture a compression rate of the rescuer 102 in an environment of the medical device 106 while the medical device 106 is located at an emergency scene 100B.

In various cases, the processor(s) 116 of the medical device 106 processes movement data 502(B) to determine a movement recognition result(s). Any suitable technology can be used to process the movement data 502(B) to determine the movement recognition result. In some examples, event detection software can recognize movement patterns (e.g., intensity, frequency, etc.), such as patterns of motions of hands of the rescuer 102 in a vertical direction (e.g., with respect to the ground surface). In various cases, the movement data 502(B) includes a frequency, a depth, a location, or a duty cycle of chest compressions administered by the rescuer 102.

At 510, for instance, the processor(s) 116 of the medical device 106 determines a characteristic of the treatment by analyzing the movement recognition result. In various examples, the processor(s) 116 determines the treatment data 140, such as a characteristic(s) of the treatment by the rescuer 102, by analyzing the movement recognition result. For example, if a movement recognition result is indicative of a movement of the hands of the rescuer 102 in a vertical direction, the processor(s) 116 may determine, from this recognition result, that a characteristic of the treatment likely includes chest compressions.

With reference to a third technique 500(C), for instance, the rescuer data 112 includes image data 502(C) representing at least a portion of the emergency scene 100B. For example, the medical device 106 may include an input device 138 in the form of a camera(s), and the treatment data 140 received by this input device 138 is in the form of image data 502(C) (e.g., one or more still images, a video, etc.). In this example, the camera(s) of the medical device 106 may capture images of at least part of the emergency scene 100B that is within a field of view of the camera(s). In some examples, the rescuer 102 is within the field of view of the camera(s) when the image data 502(C) is generated such that the image data 502(C) represents the rescuer 102 at the emergency scene 100B. In some examples, the image data 502(C) represents the patient 104, bystanders, additional medical device 114, a landscape, the sky, buildings, vehicles, or other structures and/or objects in the environment of the medical device 106, and/or the like. In some examples, the rescuer 102 may be wearing a headset and/or glasses with an outward-facing camera(s), and an input device 138 of the medical device 106 in the form of a transceiver(s) may receive the image data 502(C) from the headset and/or glasses worn by the rescuer 102.

At 512, for instance, the processor(s) 116 of the medical device 106 processes the image data 502(C) to determine an image recognition result. Any suitable image recognition technology can be used to determine an image recognition result, and a characteristic(s) of the treatment administered by the rescuer 102. In some examples, the image data 502(C) is provided as input to a trained AI model(s) and the image recognition result is received as output from the trained AI model(s).

In various examples, the trained AI model(s), such as the ML model(s), are implemented in the medical device 106 and/or another device(s). The other device(s) includes, for instance, an external device(s), such as in a cloud computing network. In various examples, the other device(s) are communicatively coupled to the medical device 106 and/or the rescuer device 108.

At 514, the processor(s) 116 of the medical device 106 determines the characteristic(s) of the treatment by analyzing the image recognition result. In some examples, the image recognition result is indicative of a size of the rescuer 102. In various cases, the characteristic(s) of the treatment administer by the rescuer 102 determined at 514 may be that the treatment is being administer to the rescuer 102 in a particular position, with various types of movements, in a particular pose, within a particular timeframe, at a particular orientation with respect to the patient 104, and/or the like, which may be useful for inferring the characteristic(s) of the treatment.

In some examples, the characteristic(s) of the treatment administered by the rescuer 102 determined at 514 may be based on a characteristic(s) of the patient 104. In those or other examples, the characteristic(s) of the treatment administered by the rescuer 102 determined at 514 may be that the treatment is being administer to the patient 104 that is obese, an adult, a child, an infant, and/or the like, which may be useful for inferring the characteristic(s) of the treatment. In some examples, the characteristic(s) of the patient 104 determined at 514 may indicate a gender of the patient 104, an age (e.g., young, middle aged, or old) of the patent 104, which may be useful for inferring the treatment administered by the rescuer 102, such as with respect to a likely medical condition of the patient 104 determined based on the characteristic(s) of the patient 104. In some examples, the characteristic(s) of the patient 104 determined at 514 may be that the patient 104 is conscious or unconscious, bleeding or not bleeding, vomiting or not vomiting, and/or the like. In some examples, the image recognition result may indicate black or dense smoke at the emergency scene 100A (e.g., from a fire), and the characteristic(s) of the patient 104 determined at 514 may be that the patient 104 is likely suffering from burns and/or a respiratory problem (e.g., smoke inhalation).

With reference to a fourth technique 500(D), for instance, the rescuer data 112 may be any suitable type of data, such as the above-described heart rate 502(A), movement data 502(B), image data 502(C), and/or other types of data including, without limitation, environmental data representing a condition of an environment of the emergency scene 100A (e.g., an environmental condition associated with air temperature, air quality, and/or flooding in the environment), physiological data representing a physiological parameter(s) of the patient 104, and/or the like.

At 516, for instance, the processor(s) 116 of the medical device 106 provides the rescuer data 112 as input to a trained AI model(s). For example, the AI model(s) include an ML model(s). At 518, for instance, the processor(s) 116 receives a characteristics of the treatment as output from the trained AI model(s). For instance, the trained AI model(s) may be configured to analyze the rescuer data 112 to predict a characteristic(s) of the treatment administered by the rescuer 102, such as any portion of the treatment data 140. By way of example, the trained AI model(s) may be configured to analyze the rescuer data 112 to predict a type(s) of treatment identified in the treatment data 140.

In various cases, the processor(s) 116 receives a characteristic(s) (e.g., a role(s)) of the rescuer 102 as output from the trained AI model(s). For instance, the rescuer data 112 is input to the trained AI model(s) and processed by the trained AI model(s). The trained AI model(s) output the characteristic(s) (e.g., the role(s)) of the rescuer 102 based on the rescuer data 112. The characteristic(s) output from the trained AI model(s) is utilized, in some examples, based on the characteristic(s) being previously, initially, and/or currently unascertainable, unknown, unidentified, unobtainable, inaccessible, etc., or any combination thereof. In alternative examples, the characteristic(s) output from the trained AI model(s) is utilized based on the characteristic(s) being previously, initially, and/or currently not readily ascertainable, known, identifiable, obtainable, accessible. In alternative or additional examples, the characteristic(s) output from the trained AI model(s) is utilized based on the characteristic(s) being difficult (e.g., slightly difficult or severely difficult) and/or time consuming to ascertain, know, identify, obtain, access, etc., or any combination thereof.

In various implementations, the rescuer data 112 includes location data, which is associated with the emergency scene. In some examples, a device(s) (e.g., a medical device 106), from among various devices, manages the rescuer data 112, which includes the location data 502(A).

In various cases, the medical device 106 accesses, from a datastore, a historical record associated with other rescuers having previously treated patients with medical issues at a location of the emergency scene 100B. In some examples, the location data is utilized by the medical device 106 to identify the location. For example, the location data includes GPS coordinates (e.g., latitude and longitude) received by a GPS receiver of the medical device 106, and, in some examples, the processor(s) 116 of the medical device 106 is configured to resolve the GPS coordinates to a residential address or a business address if the nearest address is within a threshold distance from the GPS coordinates. In some examples, the location data is received via user input provided to the medical device 106 (e.g., via a touch-sensitive display, keypad, etc.). In this example, the rescuer 102 may enter an address or cross streets that corresponds to the location of the emergency scene 100B upon arrival at the emergency scene 100B.

In some examples, the medical device 106 may have access to a datastore containing a historical record(s) of another rescuer(s) that treated patients who have been registered at a medical care facility (e.g., a hospital, a clinic, a doctor's office, etc.). For instance, the processor(s) 116 of the medical device 106 may use the location represented by the location data 502(A) to identify the historical record(s), such as a record(s) associated with the other rescuer(s). Alternatively or additionally to accessing the historical record(s) of the other rescuer(s), the medical device 106 accesses emergency medical records, such as an emergency medical record(s) (EMR(s)) associated with a registered patient residing at the location.

In some examples, the processor(s) 116 of the medical device 106 determines a characteristic of the treatment by analyzing information in the historical record. In some examples, after the historical record(s) is identified and accessed, the medical device 106 determines the characteristic(s) of the treatment by the rescuer 102 at the emergency scene 100B by analyzing information in the historical record(s). For example, the historical record(s) accessed may include information indicating other treatment administer by the rescuer 102 and/or another rescuer(s) to a registered patient(s) residing at the location of the emergency scene 100B. In some cases, the patient(s) may have a history(ies) of a medical condition(s) (e.g., a history of heart attacks, respiratory problems, etc.). In these examples, based on a reasonable likelihood that the treatment was performed on the patient 104 located at the emergency scene 100B, and/or on a patient(s) with similar symptoms, the processor(s) 116 of the medical device 106 may infer that the rescuer 102 is likely to anticipate that the treatment on the patient 104 will need to be performed. In various cases, the processor(s) 116 of the medical device 106 determines a characteristic(s) (e.g., a role(s)) of the rescuer 102 and/or another rescuer(s) by analyzing information in the historical record.

In some examples, the rescuer data 112 includes audio data. In those or other examples, the medical device 106 and/or the rescuer device 108 uses speech recognition software to recognize speech in the audio data, which may involve the utilization of automatic speech recognition and/or natural language understanding algorithms and/or models. This might allow for the sound recognition result determined at 508 to indicate something important or relevant uttered by the rescuer 102 at the emergency scene 100B, and/or by the patient 104 (if the patient 104 is conscious and talking). In some examples, the sound recognition result may indicate that the rescuer 102 and/or the patient 104 is speaking, regardless of what the rescuer 102 and/or the patient 104 is saying, which may suggest that the rescuer 102 is likely administering a type of treatment and/or that the patient 104 is likely conscious. In some examples, the sound recognition result determined at 508 may be indicative of agonal breathing, which may suggest that the patient 104 is likely experiencing a respiratory problem.

In some examples, the trained AI model(s) is configured to analyze the rescuer data 112 to predict the type of treatment based on processing a heart rate, a movement, images, and/or contextual data. In some examples, utilizing a trained AI model(s) to determine the characteristic(s) of the treatment administered by the rescuer 102 includes identifying, in the contextual data, environmental data representing a condition of an environment of the emergency scene 100A, such as an air temperature above a threshold temperature. The air temperature above the threshold temperature, for example, is utilized to determine that the rescuer 102 is administering, and/or likely to administer, treatment to the patient 104, such as with instances in which the patient 104 is experiencing heat stroke due to a heat wave in the vicinity of the emergency scene 100A. In another example, the environmental data may indicate that an air quality index (AQI) in the environment of the emergency scene 100A has fallen below a threshold AQI, and the characteristic(s) of the treatment likely includes the rescuer 102 administering treatment to the patient 104 experiencing a respiratory problem due to the smoke from a nearby wildfire, for example. In yet another example, if the contextual data includes physiological data representing a physiological parameter(s) of the patient 104 (e.g., that the patient 104 is obese), the characteristic(s) of the treatment likely includes the rescuer 102 administering treatment to the patient 104 experiencing a respiratory problem or a cardiovascular problem.

In these examples, the size of the patient 104 is inferred and utilized as part of determining whether the rescuer 102 is likely to administer treatment to the patient 104. For example, the size of the patient 104 is inferred from image data 502(C) and/or data from a nearby medical device 114, such as a mechanical chest compression device configured to measure a size of the patient 104 via an amount of adjustment of the device in order to fit the device around the torso of the patient 104, an amount of pressure detected by pressure sensors in the backplate of the device, and/or the like. In some examples, a cot or a stretcher may have pressure sensors that can detect an amount of pressure indicative of a size of the patient 104, which may be received by a transceiver(s) of the medical device 106 and used to infer that the patient 104 is obese.

FIG. 6 illustrates example techniques in a process 600 for customizing a set of alarms. As indicated in FIG. 6, the example techniques illustrated in FIG. 6 may be performed as sub-operations of block 410 of the process 400 described above.

With reference to a first technique 600(A), for instance, causing the alarm to be output to the rescuer 102, includes changing a threshold(s) used for generating an alarm(s). For example, the limit(s) (e.g., the high limit and/or low limit) associated with a particular alarm(s) of the set of alarms for the medical device 106 may be changed from a narrower range to a wider range, or vice versa, in order to adjust the sensitivity of the alarm, and/or the range between the high and low limit may be changed to a different range that is tailored to the characteristic(s) (e.g., the type of treatment) of the treatment administered by the rescuer 102.

The alarm(s), the alarm threshold(s), and/or the alarm limit(s) are managed in various ways. In some examples, the alarm(s) is generated and/or customized based on the rescuer data 112, the characteristic(s) (e.g., the role(s)) of the rescuer 102, the treatment data 140, the characteristic(s) of the treatment, and so on, or any combination thereof. In alternative or additional examples, the threshold(s) and/or the limit(s) are controlled based on the rescuer data 112 and/or a characteristic(s) (e.g., a role(s)) of the rescuer 102.

As an illustrative example, a heart rate threshold(s) of the alarm(s) is changed so that it is tailored to the treatment of the rescuer 102 who is performing chest compressions for a patient 104. In some cases, the chest compressions are performed (e.g., the treatment is being administered) by the rescuer 102 in response to the rescuer 102 detecting the patient 104 has the heart rate below the heart rate threshold. For instance, the heart rate threshold is raised to avoid distracting and/or causing alarm fatigue to the rescuer 102. In some examples, the heart rate threshold is raised to avoid causing stress to the rescuer 102 based on the rescuer 102 having a high heart rate, which may indicate the rescuer 102 is experiencing a high level of stress, confusion, concern, etc., or any combination thereof.

As another example, a set of alarms including the heart rate alarm are customized by changing the heart rate alarm limit(s) to a relatively wider range of, say, a high limit of 145 bpm and a low limit of 35 bpm. In some cases, a heart rate alarm limit(s) may be set by default to a relatively narrower range (e.g., a high limit of 125 bpm and a low limit of 50 bpm). For instance, if the characteristic(s) of the treatment is that the treatment is likely to be performed, the custom heart rate alarm limits may allow, based on the wider range of the customized limit(s), for relatively fewer distractions to the rescuer 102 while the rescuer 102 is administering the treatment.

With reference to a second technique 600(B), for instance to customize a set of alarms. the medical device 106 disables or mutes an alarm(s) that is: (i) associated with the characteristic of, but temporarily unnecessary for, the treatment administered by the rescuer 102, and/or (ii) not beneficial for continuing treatment. In various examples, the medical device 106 enables or activates an alarm(s) of the set of alarms for the medical device 106 that (i) is not associated with a determined characteristic(s) (e.g., type of treatment) of the treatment administered by the rescuer 102, or (ii) is associated with another characteristic(s) (e.g., another type(s) of treatment).

For example, if the characteristic(s) of the treatment administered by the rescuer 102 is that the treatment is being administered to the patient 104 likely having experienced a heart attack, an alarm(s) that is associated with lack of a heart rate can be disabled or muted (e.g., due to the rescuer 102 likely already being aware of the patient characteristic(s)), and/or cardiovascular-related alarms that are distracting and/or redundant for continuing treatment of the heart attack may be disabled or muted.

With reference to a third technique 600(C), for instance, the medical device 106 prioritizes output of a particular alarm(s) over another alarm(s). By way of example, by prioritizing output of the particular alarm(s) over the other alarm(s), the medical device 106 customizes a set of alarms. In some examples, if a respiratory-related alarm is prioritized over a cardiovascular-related alarm, in a scenario where both alarms are triggered at or near the same time, the processor(s) 116 of the medical device 106 causes the respiratory-related alarm that is important for informing the rescuer 102 of a treatment not currently being performed to be output. For example, the rescuer 102 performing CPR may need to be alerted regarding a respirator condition(s). In those or other examples, the medical device 106 refrains from generating the cardiovascular alarm (e.g., to be output by the medical device 106 and/or the rescuer device 108) that is distracting and/or unnecessary. In those or other examples, the processor(s) 116 of the medical device 106 suppresses the cardiovascular alarm (e.g., to be output by the medical device 106 and/or the rescuer device 108) in some way, such as disabling an audible alarm, and/or transmitting a signal therefore to the rescuer device 108, for the cardiovascular-related alarm. Alternatively or additionally, the medical device, for example, strictly outputs the respiratory-related alarm cardiovascular-related alarm on the display(s) of the medical device 106, transmits a signal therefore to the rescuer device 108. In various cases, output of the cardiovascular alarm is disabled aside from outputting of the cardiovascular alarm on the display(s) of the medical device 106 and/or on a display(s) of the rescuer device 108. In various examples, any other types of muting/enabling are performed for any types of alarm(s) to eliminate distractions to the rescuer 102, as preferred.

With reference to a fourth technique 600(D), for instance, the medical device 106 changes a tone and/or intensity level of an audible alarm(s). In some examples, to change the tone and/or intensity level of the audible alarm(s), the medical device 106 customizes a set of alarms of the medical device 106 and/or transmits a signal therefore to the rescuer device 108. In those or other examples, the medical device 106 changes a tone and/or an intensity level associated with an alarm(s) of the set of alarms, and/or transmits a signal therefore to the rescuer device 108. In those or other examples, the medical device 106 changes the tone of an alarm(s) (e.g., the cardiovascular alarm) to another tone that is less jarring and/or noticeable to a rescuer 102 at the emergency scene where the patient 104 is located, and/or transmits a signal to change the tone/intensity level to the rescuer device 108. In various cases, the tone an alarm(s) (e.g., the cardiovascular alarm) is deprioritized to another tone that is more subtle so that sensory overload of the rescuer 102 is mitigated.

As another example, an intensity level at which a deprioritized alarm(s) is to be output via the output device(s) 124 and/or the rescuer device 108 is decreased to make the deprioritized alarm(s) less onerous and distracting to the rescuer 102. For instance, an intensity level at which a deprioritized alarm(s) is to be output via the output device(s) 124 and/or the rescuer device 108 is decreased to make the deprioritized alarm(s) less conspicuous. In various cases, the medical device 106 decreases an intensity level at which the cardiovascular alarm is to be output via the output device(s) 124 and/or the rescuer device 108 to make the deprioritized alarm(s) less onerous to the rescuer 102 administering the treatment.

With reference to a fifth technique 600(E), for instance, the medical device 106 changes a color, a size, a shape, and/or an orientation of a visual indicator for an alarm(s). Alternatively or additionally, the medical device 106 transmits a signal to the rescuer device 108 to change a color, a size, a shape, and/or an orientation of a visual indicator for an alarm(s). In some examples, the medical device 106 customizes a set of alarms of the medical device 106, and/or the rescuer device 108, the customizing including changing the color, the size, the shape, and/or the orientation of the visual indicator(s). In those or other examples, the visual indicator(s) for which the color, the size, the shape, and/or the orientation is to be presented via a user interface on the display(s) of the medical device 106 and/or presented by a user interface on a display(s) of the rescuer device 108. For instance, a visual indicator for a deprioritized alarm(s) may be changed from a red color to a different, more subtle, color and is reduced in size so that it is less distracting. In some examples, the medical device 106 changes the color, the size, the shape, and/or the orientation of the visual indicator(s) associated with the cardiovascular alarm.

With reference to a sixth technique 600(F), for instance, the medical device 106 changes a location on the user interface where the visual indicator is to be presented for an alarm(s). For example, a visual indicator associated with the alarm(s) is presented at or near a side, edge, or periphery of the display(s) of one or more of the medical device 106 and/or the rescuer device 108, and away from a center of the display, to make it less distracting. In some examples, the medical device 106 and/or the rescuer device 108 presents a visual indicator(s) associated with the cardiovascular alarm at or near a side, edge, or periphery of the display.

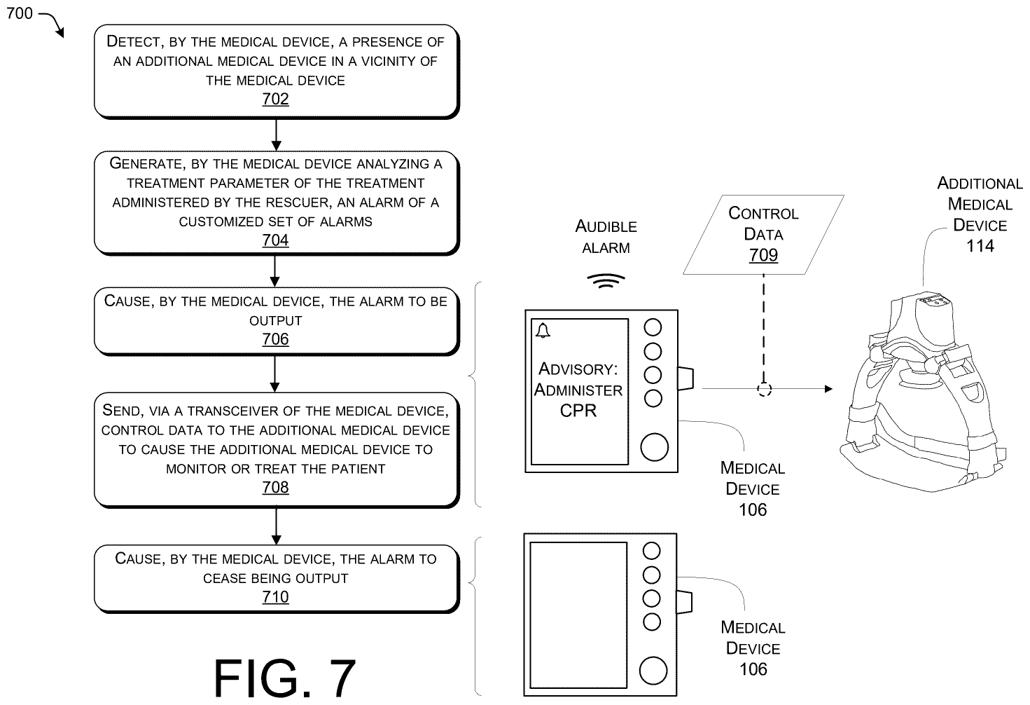

FIG. 7 illustrates an example process 700 for managing alarms of a medical device in the context of controlling an additional medical device(s) in a vicinity of the medical device. At 702, for instance, a medical device 106 detects a presence of an additional medical device 114 in a vicinity of the medical device 106. The detection at 702 can be implemented in various ways, such as by using a camera(s) of the medical device 106 to capture one or more images (e.g., still image(s) video, etc.) of the additional medical device 114 and processing the resulting image data to detect the additional medical device 114 from the captured image(s). As another example, as described above, a communication and/or pairing procedure may be utilized between the medical device 106 and the additional medical device 114 at an emergency scene 100A. For instance, the detection at 702 may be based at least in part on such a communication and/or pairing procedure. For instance, the detection at 702 may be based at least in part on such the medical device 106 detecting an electrical characteristic(s) of the additional medical device 114.

At 704, for instance, the medical device 106 generates an alarm(s) of a customized set of alarms by analyzing a treatment parameter of the treatment administered by the rescuer, an alarm of a customized set of alarms. In some examples, the processor(s) 116 of the medical device 106 is configured to generate the alarm(s) at 704 by processing the treatment parameter(s) of the treatment administered by the rescuer 102 to the patient 104. In various cases, the processor(s) 116 use the treatment parameter(s) of the treatment administered by the rescuer 102 to determine whether to generate the alarm(s) or refrain from generating the alarm(s). In some examples, the alarm(s) generated at 704 is associated with a medical condition of the patient 104, such as a heart-related medical condition.

In alternative or additional examples, the medical device 106 generates an alarm(s) of a customized set of alarms by analyzing a treatment parameter(s) of a treatment(s) administered by another rescuer(s). In those or other examples, the medical device 106 generates an alarm(s) of a customized set of alarms by analyzing a treatment parameter(s) of a treatment(s) administered by the rescuer 102 and/or the other rescuer(s), using the medical device 106, the rescuer device 108, and/or the additional medical device 114. The alarm(s) are customized to avoid distraction to the rescuer 102 and/or the other rescuer(s). In some cases, the medical device 106 divides alarms between devices of the rescuer 102 and the other rescuer(s). For example, the alarms being divided (e.g., output by different devices) include the alarm being output to the rescuer 102 and another alarm being output to the other rescuer.

At 706, the medical device 106 causes the alarm(s) to be output. In some examples, the processor(s) 116 of the medical device 106 is configured to cause the alarm(s) to be output at 706 via an output device(s) 124 of the medical device 106 and/or by the rescuer device 108 (e.g., based on a signal from the medical device 106). The alarm(s) output at 706 can be a visual alarm, an audible alarm, a tactile alarm, or any other suitable type of alarm that is designed to alert someone (e.g., a rescuer 102) as to a medical condition of the patient 104. In an illustrative example, the alarm(s) output at 706 can be a VF/VT alarm.

At 708, in response to the causing the alarm(s) to be output at 706, the medical device 106 sends, via a transceiver(s) 122 of the medical device 106, control data 709 to the additional medical device 114 to cause the additional medical device 114 to monitor or treat the patient 104. In the example of FIG. 7, the control data 709 may instruct the additional medical device 114 to begin chest compressions, since the additional medical device 114 depicted in FIG. 7 is a mechanical chest compression device.

In some examples, data that includes the control data 709 and/or other data is utilized by the medical device 106 to customize another alarm(s) of the additional medical device 114. For instance, the medical device 106 communicates the data to the additional medical device 114. In various cases, the other alarm(s) of the additional medical device 114 is customized in a similar way as, or a different way from, the alarm(s) of the medical device 106 and/or the rescuer device 108 that are customized.

At 710, for instance, in response to the sending the control data 709 to the additional medical device 114, the medical device 106 causes the alarm(s) to cease being output. For example, if the alarm(s) is designed to alert the rescuer 102 that the patient 104 is in need of CPR treatment, and if the medical device 106 can automatically control the additional medical device 114 to commence CPR, or at least part of the CPR treatment, then the rescuer 102 may not need to know about the alarm(s) anymore, since the additional medical device 114 has addressed the alarm by commencing related treatment with respect to the patient 104.

In some examples, data that includes the control data 709 and/or other data is utilized by the medical device 106 to cause the other alarm(s) of the additional medical device 114 to cease being output. For instance, the medical device 106 communicates the data to the additional medical device 114. In various cases, output of the other alarm(s) of the additional medical device 114 are caused to cease a similar way as, or a different way from, the alarm(s) of the medical device 106 that is are caused to cease being output. The medical device 106, the rescuer device 108, and/or the additional medical device 114 communicate to manage alarm(s) and/or limit(s), thereof.

Other ways of suppressing alarms to mitigate alarm fatigue are contemplated here, such as allowing a rescuer to issue a voice command to stop an ongoing alarm. In some examples, if such a voice command is detected via a microphone(s) of the medical device 106, the rescuer device 108, and/or the additional medical device 114 and using speech processing software to recognize the intent of the voice command, the medical device 106 causes the ongoing alarm to cease being output, and/or transmits a signal(s) to the rescuer device 108 and/or the additional medical device 114 therefore. Additionally, or alternatively, interactive elements are presented on a touch-sensitive display of the medical device 106 the rescuer device 108 and/or the additional medical device 114. Based on the rescuer 102 selecting an interactive element associated with an ongoing alarm to disable the alarm, for instance, alarm fatigue may be mitigated through a simple interaction with a touch-sensitive display.

In some examples, the medical device 106, the rescuer device 108 and/or the additional medical device 114 are configured to notify personnel qualified to address an alarm in response to generating and/or outputting the alarm. For example, the medical device 106, the rescuer device 108 and/or the additional medical device 114 are configured to send an electronic mail (email) message, a text message, a push notification, and/or the like to a qualified personnel device(s), such as a device(s) of an additional rescuer or rescue team in transit to the emergency scene where the patient 104 is located. In some examples, the medical device 106, the rescuer device 108 and/or the additional medical device 114 are configured to notify a remote system about a generated alarm, wherein the remote system is configured to monitor deployed medical devices including the medical device 106. In this manner, the remote system can be made aware of any medical device in the field that has generated an alarm.

FIG. 8 illustrates an example process 800 for managing alarms of a medical device based on a medication administered by a rescuer at an emergency scene. At 802, for instance, a medical device 106 receives medication data representing a medication administered by the rescuer (e.g., a rescuer 102) and to a patient 104 at an emergency scene 100A. In some cases, the medical device 106 receives such medication data via user input to the medical device 106 (e.g., the rescuer 102 may enter the medication data via a touch-sensitive display of the medical device 106, a keypad of the medical device 106, and/or the like). In various examples, the medication data is received from (e.g., communicated by) an additional medical device 114 (e.g., a medication-administering device, such as an IV pump) via a transceiver(s) 122 of the medical device 106. In some cases, this occurs after the medical device 106 and the additional medical device 114 are paired at the emergency scene 100A, as described above.

At 804, for instance, the medical device 106 presents, via a touch-sensitive display of the medical device 106, a potential alarm list that includes a subset of a customized set of alarms. In various cases, the subset is associated with the medication administered by the rescuer 102 as part of the treatment by the rescuer 102 and to the patient 104.

At 806, for instance, the medical device 106 receives, via the touch-sensitive display, a deselection(s) of an alarm(s) in the potential alarm list. For example, as illustrated in FIG. 8, an operator of the medical device 106 (e.g., the rescuer 102) deselects an interactive element associated with “Alarm B” in the potential alarm list.

At 808, for instance, the medical device 106 disables the selected alarm(s). In some cases, the medical device 106 disables the alarm(s) selected via user input to the touch-sensitive display, in response to receiving the deselection(s). For example, “Alarm B,” which is represented by a circle without an “X” as illustrated in FIG. 8, that is initially enabled is disabled if the operator (e.g., the rescuer 102) deselects “Alarm B” in the potential alarm list. Accordingly, the process 800 allows an operator of the medical device 106, such as the rescuer 102, to manage the alarms in a way that mitigates alarm fatigue by disabling certain alarms the operator does not deem beneficial for monitoring and/or treating the patient 104 at the emergency scene 100A.

In various implementations, any types of visual indicators are utilized by the medical device 106, such as the touch-sensitive display, to adjust the alarm(s) and/or the boundary(ies), such as the boundary(ies) 126 as discuss above with reference to FIG. 1A. For example, “Alarm B” that is disabled, is enabled (e.g., or re-enabled) if the operator (e.g., the rescuer 102) selects (e.g., or re-selects) “Alarm B” in the potential alarm list. In some cases, the medical device 106, such as the touch-sensitive display, presents the setting(s) mode 130 enabling the boundary(ies) 126 to be set, based on an alarm(s) being selected. For instance, user input to the touch-sensitive display is used to select, toggle, enter, etc., or any combination thereof, a value(s) of the boundary(ies) 126 to be used for controlling the alarm 134. Accordingly, the operator, such as the rescuer 102, manages the alarm(s) so that it is, and/or will be at a potential future occurrence of operation of the medical device 106, less distracting during a time(s) in which the rescuer 102 is treating the patient 104 for a related condition. The rescuer 102, for example, providing chest compressions will be able to engage and concentrate on the treatment more fully, without the alarm distracting the rescuer 102 regarding the heart rate of the patient 104 being low, of which the rescuer 102 is aware and in the process of treating.

In various examples, management of the alarm(s) and/or the limit(s), such as by selecting and/or setting one or more characteristic(s) thereof, is performed by the medical device 106, the rescuer device 108, the additional medical device 114, etc., or any combination thereof, in a similar way as, or a different way from, the management via the medical device 106, as discussed above. For example, the rescuer device 108 outputs, via a touch-sensitive display, a selection(s) of an alarm(s) in a potential alarm list utilized to control the alarm(s) and/or the limit(s). In some examples, the medical device 106, the rescuer device 108, the additional medical device 114 is identified, such as by a selection(s) via user input to any of the device(s) (e.g., the medical device 106, the rescuer device 108, the additional medical device 114), as the default device to overrule the remaining device(s). In those or other examples, selection of the medical device 106, the rescuer device 108, and/or the additional medical device 114 as the default device is automated and/or utilized as an override(s) for a previous setting(s), such as via analysis and output by the trained AI model.

FIG. 9 illustrates an example process 900 for preconfiguring the alarms of a medical device and simulating the alarms via an alarm and boundary configuration user interface presented on a user device. At 902, for instance, a processor(s) of a user device 903 causes an alarm and boundary configuration user interface 905 to be presented via a display of the user device 903. The alarm and boundary configuration user interface 905 allows a user (e.g., a caregiver, paramedic, etc.) to preconfigure a set of alarms that a medical device 106 is configured to output at emergency scenes. The set of alarms, for example, are output based on a rescuer-related metric(s) identified for the rescuer 102 that falls within a boundary(ies). The set of alarms, for example, are not output based on a rescuer-related metric(s) identified for the rescuer 102 that exceeds a boundary(ies). In some examples, a user device(s), such as the user device 903, includes the rescuer device 108, another device, or any combination thereof.

At 904, for instance, the user device 903 receives a selection(s) associated with the alarm and boundary configuration user interface 905 to control an alarm(s), an alarm limit(s), a rescuer-related metric boundary(ies), or any combination thereof. By way of example, the user of the user device 903 interacts, such as via user input to a touch screen of the user device 903, with interactive elements (e.g., drop down menus, radio buttons, etc.) presented via the alarm and boundary configuration user interface 905. In some cases, the user input is identified by the user device 903 and utilized by the user device 903 to enable or disable certain alarms, adjust the alarm limits associated with certain alarms, adjust the boundary(ies) utilized for management of the alarm(s) based on the rescuer-related metric(s), etc.

At 906, for instance, the user device 903 causes the medical device 106 to preconfigure a set of alarms and/or a set of rescuer-related metric boundaries based on the selection(s). By way of example, the set of alarms being preconfigured includes a set of alarms that a medical device 106 is configured to output at emergency scenes). In such an example or another example, the set of rescuer-related metric boundary(ies) being preconfigured includes a set of boundaries associated with a set of metrics for a set of parameters that are identifiable by a medical device 106, a rescuer device 108, an additional device, etc., or any combination thereof. In some cases, the set of alarms and/or the set of boundary(ies) are preconfigured based on a selection(s), such as the selection(s) received at 904. By way of example, the user device 903 may send data to the medical device 106 via a communication network(s) 907 for preconfiguring the set of alarms and/or the set of boundary(ies) based on the selection(s) received at 904.

At 908, for instance, the user device 903 receives a selection(s) associated with the alarm and boundary configuration user interface 905 to control a simulation(s). By way of example, the selection(s) received at 908 involve the user device 903 selecting, via user input, an interactive element(s) (e.g., a “simulate alarms” button) to simulate one or more of the alarms of the set of alarms for the medical device 106.

At 910, for instance, a processor(s) of the user device 903 causes the user device to output alarm(s) as a simulation of the medical device outputting the alarm(s). By way of example, the user device 903 outputs the set of alarms in accordance with the selections received at 908, as a simulation of the medical device 106 outputting the set of alarms. In various cases, the user device 903 outputs the set of alarms in accordance with the selections received at 908 as a simulation of the medical device 106 outputting the set of alarms. For example, the user device 903 outputs audible alarms via a speaker(s) of the user device 903 as they would sound if the audible alarms were output via a speaker(s) of the medical device 106. In some cases, the user device 903 presents visual alarms via a display(s) of the user device 903 as they would look if the visual alarms were presented via a display(s) of the medical device 106. In this way, a user of the user device 903 can preconfigure alarms and simulate the alarms on the user device 903 to ensure that the alarms are preconfigured how the user wants them to be. The user can utilize the simulations to control alarm(s) of the medical device 106, thereby reducing alarm fatigue.

The user can use the interface 905 to adjust the boundary(ies) so that any number of the alarm(s) are output in any number of ways for a corresponding boundary(ies). For example, more than one of the boundary(ies) can be utilized to adjust any of the alarm(s). In some cases, a first boundary with a narrowest range of a group of boundaries is set to configure the alarm to have a volume level; a second boundary with a somewhat wider range than the first boundary is set so the alarm has another relatively quieter volume level than for the first boundary; and a third boundary with a somewhat wider range than the second boundary is set so the alarm has another relatively quieter volume level than for the second boundary. The rescuer 102, in some instances, is able to set the volume of the alarm to decrease as the patient parameter falls within relatively more extreme levels, so that the rescuer 102 being already alerted by the alarm and/or aware of the patient parameter and treating the patient 104 is not distracted as the seriousness of the condition of the patient 104 escalates and as the rescuer is administering treatment. In various embodiments, any number of characteristics (e.g., the volume) associated with any number of the alarm(s) and/or any number of the boundary(ies) are able to be set by the interface 905, for any number and/or types of the rescuer-related metric(s).

In some examples, an AI model(s) learns or is trained to recognize how, or in what ways, certain caregivers or paramedics could improve their treatment (such as the quality of ventilation), and the AI model(s) may suggest a customized set of alarms via the alarm and boundary configuration user interface 905 based on the improvement suggestions for the user group.

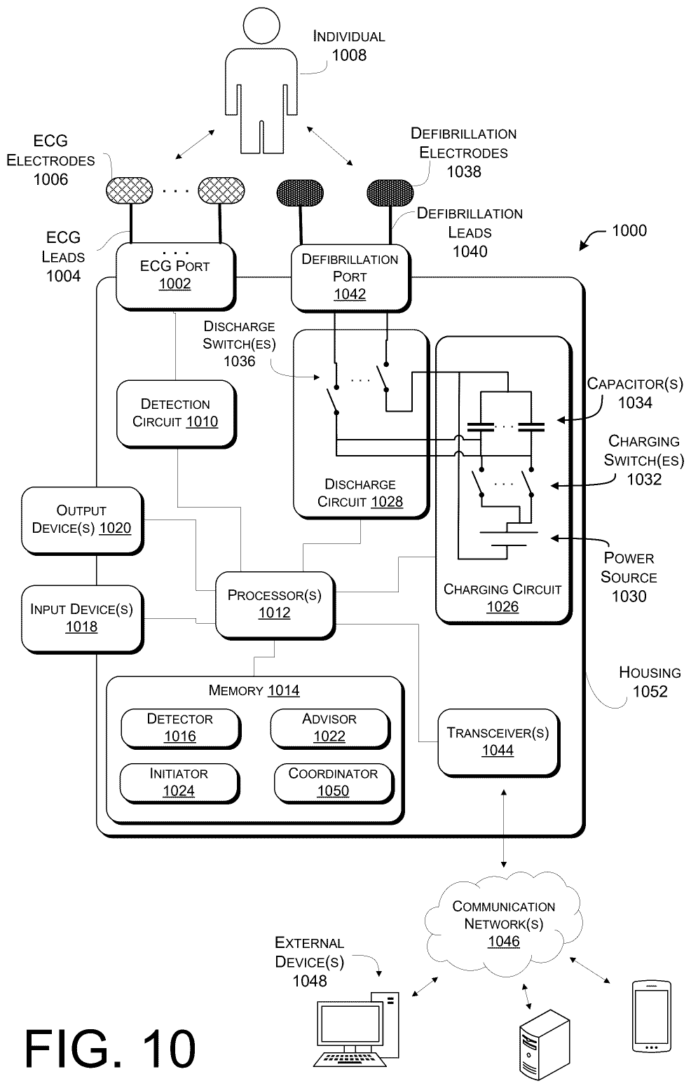

FIG. 10 illustrates an example of an external defibrillator 1000 configured to perform various functions described herein. For example, the external defibrillator 1000 is the medical device 106 or an additional medical device(s) 114 described above with reference to FIGS. 1A to 3.

The external defibrillator 1000 includes an ECG port 1002 connected to multiple ECG leads 1004. In some cases, the ECG leads 1004 are removeable from the ECG port 1002. For instance, the ECG leads 1004 are plugged into the ECG port 1002. The ECG leads 1004 are connected to ECG electrodes 1006, respectively. In various implementations, the ECG electrodes 1006 are disposed on different locations on an individual 1008. A detection circuit 1010 is configured to detect relative voltages between the ECG electrodes 1006. These voltages are indicative of the electrical activity of the heart of the individual 1008.

In various implementations, the ECG electrodes 1006 are in contact with the different locations on the skin of the individual 1008. In some examples, a first one of the ECG electrodes 1006 is placed on the skin between the heart and right arm of the individual 1008, a second one of the ECG electrodes 1006 is placed on the skin between the heart and left arm of the individual 1008, and a third one of the ECG electrodes 1006 is placed on the skin between the heart and a leg (either the left leg or the right leg) of the individual 1008. In these examples, the detection circuit 1010 is configured to measure the relative voltages between the first, second, and third ECG electrodes 1006. Respective pairings of the ECG electrodes 1006 are referred to as “leads,” and the voltages between the pairs of ECG electrodes 1006 are known as “lead voltages.” In some examples, more than three ECG electrodes 1006 are included, such that 5-lead or 12-lead ECG signals are detected by the detection circuit 1010.

The detection circuit 1010 includes at least one analog circuit, at least one digital circuit, or a combination thereof. The detection circuit 1010 receives the analog electrical signals from the ECG electrodes 1006, via the ECG port 1002 and the ECG leads 1004. In some cases, the detection circuit 1010 includes one or more analog filters configured to filter noise and/or artifact from the electrical signals. The detection circuit 1010 includes an analog-to-digital (ADC) in various examples. The detection circuit 1010 generates a digital signal indicative of the analog electrical signals from the ECG electrodes 1006. This digital signal can be referred to as an “ECG signal” or an “ECG.”

In some cases, the detection circuit 1010 further detects an electrical impedance between at least one pair of the ECG electrodes 1006. For example, the detection circuit 1010 includes, or otherwise controls, a power source that applies a known voltage (or current) across a pair of the ECG electrodes 1006 and detects a resultant current (or voltage) between the pair of the ECG electrodes 1006. The impedance is generated based on the applied signal (voltage or current) and the resultant signal (current or voltage). In various cases, the impedance corresponds to respiration of the individual 1008, chest compressions performed on the individual 1008, and other physiological states of the individual 1008. In various examples, the detection circuit 1010 includes one or more analog filters configured to filter noise and/or artifact from the resultant signal. The detection circuit 1010 generates a digital signal indicative of the impedance using an ADC. This digital signal can be referred to as an “impedance signal” or an “impedance.”

The detection circuit 1010 provides the ECG signal and/or the impedance signal one or more processors 1012 in the external defibrillator 1000. In some implementations, the processor(s) 1012 includes a central processing unit (CPU), a graphics processing unit (GPU), both CPU and GPU, or other processing unit or component known in the art.

The processor(s) 1012 is operably connected to memory 1014. In various implementations, the memory 1014 is volatile (such as random access memory (RAM)), non-volatile (such as read only memory (ROM), flash memory, etc.) or some combination of the two. The memory 1014 stores instructions that, when executed by the processor(s) 1012, causes the processor(s) 1012 to perform various operations. In various examples, the memory 1014 stores methods, threads, processes, applications, objects, modules, any other sort of executable instruction, or a combination thereof. In some cases, the memory 1014 stores files, databases, or a combination thereof. In some examples, the memory 1014 includes, but is not limited to, RAM, ROM, electrically erasable programmable read-only memory (EEPROM), flash memory, or any other memory technology. In some examples, the memory 1014 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 1012 and/or the external defibrillator 1000. In some cases, the memory 1014 at least temporarily stores the ECG signal and/or the impedance signal.

In various examples, the memory 1014 includes a detector 1016, which causes the processor(s) 1012 to determine, based on the ECG signal and/or the impedance signal, whether the individual 1008 is exhibiting a particular heart rhythm. For instance, the processor(s) 1012 determines whether the individual 1008 is experiencing a shockable rhythm that is treatable by defibrillation. Examples of shockable rhythms include ventricular fibrillation (VF) and ventricular tachycardia (V-Tach). In some examples, the processor(s) 1012 determines whether any of a variety of different rhythms (e.g., asystole, sinus rhythm, atrial fibrillation (AF), etc.) are present in the ECG signal.

The processor(s) 1012 is operably connected to one or more input devices 1018 and one or more output devices 1020. Collectively, the input device(s) 1018 and the output device(s) 1020 function as an interface between a user and the defibrillator 1000. The input device(s) 1018 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 1020 includes at least one of a display, a speaker, a haptic output device, a printer, or any combination thereof. In various examples, the processor(s) 1012 causes a display among the input device(s) 1018 to visually output a waveform of the ECG signal and/or the impedance signal. In some implementations, the input device(s) 1018 includes one or more touch sensors, the output device(s) 1020 includes a display screen, and the touch sensor(s) are integrated with the display screen. Thus, in some cases, the external defibrillator 1000 includes a touchscreen configured to receive user input signal(s) and visually output physiological parameters, such as the ECG signal and/or the impedance signal.

In some examples, the memory 1014 includes an advisor 1022, which, when executed by the processor(s) 1012, causes the processor(s) 1012 to generate advice and/or control the output device(s) 1020 to output the advice to a user (e.g., a rescuer). In some examples, the processor(s) 1012 provides, or causes the output device(s) 1020 to provide, an instruction to perform CPR on the individual 1008. In some cases, the processor(s) 1012 evaluates, based on the ECG signal, the impedance signal, or other physiological parameters, CPR being performed on the individual 1008 and causes the output device(s) 1020 to provide feedback about the CPR in the instruction. According to some examples, the processor(s) 1012, upon identifying that a shockable rhythm is present in the ECG signal, causes the output device(s) 1020 to output an instruction and/or recommendation to administer a defibrillation shock to the individual 1008.

The memory 1014 also includes an initiator 1024 which, when executed by the processor(s) 1012, causes the processor(s) 1012 to control other elements of the external defibrillator 1000 in order to administer a defibrillation shock to the individual 1008. In some examples, the processor(s) 1012 executing the initiator 1024 selectively causes the administration of the defibrillation shock based on determining that the individual 1008 is exhibiting the shockable rhythm and/or based on an input from a user (received, e.g., by the input device(s) 1018. In some cases, the processor(s) 1012 causes the defibrillation shock to be output at a particular time, which is determined by the processor(s) 1012 based on the ECG signal and/or the impedance signal.

The processor(s) 1012 is operably connected to a charging circuit 1026 and a discharge circuit 1028. In various implementations, the charging circuit 1026 includes a power source 1030, one or more charging switches 1032, and one or more capacitors 1034. The power source 1030 includes, for instance, a battery. The processor(s) 1012 initiates a defibrillation shock by causing the power source 1030 to charge at least one capacitor among the capacitor(s) 1034. For example, the processor(s) 1012 activates at least one of the charging switch(es) 1032 in the charging circuit 1026 to complete a first circuit connecting the power source 1030 and the capacitor to be charged. Then, the processor(s) 1012 causes the discharge circuit 1028 to discharge energy stored in the charged capacitor across a pair of defibrillation electrodes 1038, which are in contact with the individual 1008. For example, the processor(s) 1012 deactivates the charging switch(es) 1032 completing the first circuit between the capacitor(s) 1034 and the power source 1030, and activates one or more discharge switches 1036 completing a second circuit connecting the charged capacitor 1034 and at least a portion of the individual 1008 disposed between defibrillation electrodes 1038.

The energy is discharged from the defibrillation electrodes 1038 in the form of a defibrillation shock. For example, the defibrillation electrodes 1038 are connected to the skin of the individual 1008 and located at positions on different sides of the heart of the individual 1008, such that the defibrillation shock is applied across the heart of the individual 1008. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 J or greater with a duration of about 0.015 seconds. In some cases, the defibrillation shock has a multiphasic (e.g., biphasic) waveform. The discharge switch(es) 1036 are controlled by the processor(s) 1012, for example. In various implementations, the defibrillation electrodes 1038 are connected to defibrillation leads 1040. The defibrillation leads 1040 are connected to a defibrillation port 1042, in implementations. According to various examples, the defibrillation leads 1040 are removable from the defibrillation port 1042. For example, the defibrillation leads 1040 are plugged into the defibrillation port 1042.

In various implementations, the processor(s) 1012 is operably connected to one or more transceivers 1044 that transmit and/or receive data over one or more communication networks 1046. For example, the transceiver(s) 1044 includes a network interface card (NIC), a network adapter, a local area network (LAN) adapter, or a physical, virtual, or logical address to connect to the various external devices and/or systems. In various examples, the transceiver(s) 1044 includes any sort of wireless transceivers capable of engaging in wireless communication (e.g., radio frequency (RF) communication). For example, the communication network(s) 1046 includes one or more wireless networks that include a 3rd Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 1044 includes other wireless modems, such as a modem for engaging in WI-FI®, WIGIG®, WIMAX®, BLUETOOTH®, NFC, radio frequency identification (RFID), or infrared communication over the communication network(s) 1046.

The defibrillator 1000 is configured to transmit and/or receive data (e.g., ECG data, impedance data, data indicative of one or more detected heart rhythms of the individual 1008, data indicative of one or more defibrillation shocks administered to the individual 1008, etc.) with one or more external devices 1048 via the communication network(s) 1046. The external devices 1048 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 1046. In some examples, the external device(s) 1048 is located remotely from the defibrillator 1000, such as at a remote clinical environment (e.g., a hospital). According to various implementations, the processor(s) 1012 causes the transceiver(s) 1044 to transmit data to the external device(s) 1048. In some cases, the transceiver(s) 1044 receives data from the external device(s) 1048 and the transceiver(s) 1044 provide the received data to the processor(s) 1012 for further analysis.

In some cases, the external device(s) 1048 include one or more medical devices. According to various implementations, the memory 1014 further includes a coordinator 1050 which, when executed by the processor(s) 1012, causes the processor(s) 1012 to coordinate with the external device(s) 1048, such as by administering therapy (e.g., defibrillation, pacing, etc.) to a subject based on communication between the defibrillator 1000 and the external device(s) 1048, as described herein. In some implementations, the processor(s) 1012, when executing the coordinator 1050, receives data from the external device(s) 1048, analyzes the data to determine a control parameter(s), and administers therapy (e.g., defibrillation, pacing, etc.) in accordance with the control parameter(s), as described herein. In some implementations, the processor(s) 1012, when executing the coordinator 1050, determines a parameter associated with the therapy being administered by the defibrillator 1000, such as a physiological parameter of the individual 1008, determines, by analyzing the parameter, a control parameter for controlling administration of therapy to the individual 1008 by the external device(s) 1048, and sends data (e.g., via the transceiver(s) 1044) to the external device(s) 1048, the data representing the control parameter. In general, the coordinator 1050, when executed by the processor(s) 1012, may cause the processor(s) 1012 to perform any of the processes 300-800 described herein.

In various implementations, the external defibrillator 1000 also includes a housing 1052 that at least partially encloses other elements of the external defibrillator 1000. For example, the housing 1052 encloses the detection circuit 1010, the processor(s) 1012, the memory 1014, the charging circuit 1026, the transceiver(s) 1044, or any combination thereof. In some cases, the input device(s) 1018 and output device(s) 1020 extend from an interior space at least partially surrounded by the housing 1052 through a wall of the housing 1052. In various examples, the housing 1052 acts as a barrier to moisture, electrical interference, and/or dust, thereby protecting various components in the external defibrillator 1000 from damage.

In some implementations, the external defibrillator 1000 is an automated external defibrillator (AED) operated by an untrained user (e.g., a bystander, layperson, etc.) and can be operated in an automatic mode. In automatic mode, the processor(s) 1012 automatically identifies a rhythm in the ECG signal, makes a decision whether to administer a defibrillation shock, charges the capacitor(s) 1034, discharges the capacitor(s) 1034, or any combination thereof. In some cases, the processor(s) 1012 controls the output device(s) 1020 to output (e.g., display) a simplified user interface to the untrained user. For example, the processor(s) 1012 refrains from causing the output device(s) 1020 to display a waveform of the ECG signal and/or the impedance signal to the untrained user, in order to simplify operation of the external defibrillator 1000.

In some examples, the external defibrillator 1000 is a monitor-defibrillator utilized by a trained user (e.g., a clinician, an emergency responder, etc.) and can be operated in a manual mode or the automatic mode. When the external defibrillator 1000 operates in manual mode, the processor(s) 1012 cause the output device(s) 1020 to display a variety of information that may be relevant to the trained user, such as waveforms indicating the ECG data and/or impedance data, notifications about detected heart rhythms, and the like.

### EXAMPLE CLAUSES

The following clauses provide various examples of the present disclosure. However, the scope of the present disclosure should not be interpreted as being limited to any of the clauses listed below.
1: A system, including: a wearable device configured to be worn by a rescuer, the wearable device including: a sensor configured to detect a heart rate of the rescuer; and a first transceiver configured to: transmit a communication signal indicating the heart rate of the rescuer; and receive a communication signal indicating an alarm; and an output device configured to output the alarm to the rescuer; and a medical device, including: a measurement circuit configured to detect an electrocardiogram (ECG) of a patient; a second transceiver configured to: receive the communication signal indicating the heart rate of the rescuer; and transmit the communication signal indicating the alarm; and a processor configured to: determine that the ECG is indicative of an arrhythmia; in response to determining that the ECG is indicative of the arrhythmia, generate the alarm; determine that the heart rate of the rescuer is below a threshold; and in response to determining that the heart rate of the rescuer is below the threshold, cause the second transceiver to transmit the communication signal indicating the alarm.
2: The system of clause 1, wherein the output device is configured to output the alarm to the rescuer by: outputting a projection indicating the alarm; outputting a hologram indicating the alarm; or outputting an augmented reality (AR) alarm.
3: The system of clause 1 or 2, wherein the wearable device includes a smart watch, and wherein the output device includes a speaker configured to audibly output the alarm.
4: The system of any of clauses 1 to 3, the heart rate being detected by the sensor at a first time, wherein the sensor is further configured to detect the heart rate of the rescuer at a second time, wherein the first transceiver is further configured to transmit a communication signal indicating the heart rate of the rescuer at the second time, wherein the second transceiver is further configured to receive the communication signal indicating the heart rate of the rescuer at the second time, and wherein the processor is further configured to: determine that the heart rate of the rescuer at the second time is above the threshold; and in response to determining that the heart rate of the rescuer at the second time is above the threshold, cause the second transceiver to transmit a communication signal causing the wearable device to refrain from outputting the alarm.
5: A medical device, including: a sensor configured to detect a physiological parameter of a patient; a transceiver configured to receive data indicating a parameter of a rescuer; a processor configured to: generate an alarm by analyzing the physiological parameter of the patient; compare the parameter of the rescuer to a threshold; and in response to comparing the parameter of the rescuer to the threshold, cause the alarm to be output to the rescuer.
6: The medical device of clause 5, wherein the physiological parameter of the patient includes a heart rate, an electrocardiogram (ECG), a respiration rate, capnograph, a blood pressure, a transthoracic impedance, a temperature, or a blood oxygenation.
7: The medical device of clause 5 or 6, wherein the physiological parameter of the patient includes a first physiological parameter detected by the sensor at a first time, and wherein the processor is further configured to: determine that the first physiological parameter at a second time is above the threshold; and in response to determining that the first physiological parameter at the second time is above the threshold, cause the transceiver to transmit a communication signal causing a wearable device configured to be worn by the rescuer to refrain from outputting the alarm.
8: The medical device of any of clauses 5 to 7, wherein the parameter of the rescuer includes a blood pressure, a respiration rate, a temperature, an acceleration, a location, a vocal sound, or a pupil movement.
9: The medical device of any of clauses 5 to 8, wherein the parameter of the rescuer includes a level of body movement detected by a motion sensor of a wearable device worn by the rescuer, a volume of a vocal sound detected by an audio recorder of the wearable device, or a pitch of the vocal sound.
10: The medical device of any of clauses 5 to 9, wherein the parameter of the rescuer corresponds to a predicted stress level of the rescuer.
11: A method, including: detecting a physiological parameter of a patient; generating an alarm by analyzing the physiological parameter of the patient; receiving data indicating a parameter of a rescuer; comparing the parameter of the rescuer to a threshold; in response to comparing the parameter of the rescuer to the threshold, causing the alarm to be output to the rescuer.
12: The method of clause 11, wherein the physiological parameter of the patient includes a heart rate, an electrocardiogram (ECG), a respiration rate, a capnograph, a blood pressure, a transthoracic impedance, a temperature, or a blood oxygenation.
13: The method of clause 11 or 12, wherein generating the alarm by analyzing the physiological parameter of the patient includes determining that the physiological parameter of the patient is indicative of an emergency medical condition.
14: The method of any of clauses 11 to 13, wherein the parameter of the rescuer includes a heart rate, a blood pressure, a respiration rate, a temperature, an acceleration, a location, a volume of a vocal sound, a pitch of the vocal sound, or a pupil movement.
15: The method of any of clauses 11 to 14, wherein receiving the data indicating the parameter of the rescuer includes: receiving the data indicating the parameter of the rescuer from a portable computing device including smart glasses, a smart watch, or a recording device.
16: The method of any of clauses 11 to 15, further including: identifying a role of the rescuer at a rescue scene, the rescuer being one of multiple rescuers at the rescue scene; and determining that the role of the rescuer is associated with the alarm, wherein causing the alarm to be output to the rescuer is further in response to determining that the role of the rescuer is associated with the alarm.
17: The method of any of clauses 11 to 16, the rescuer being a first rescuer at a rescue scene, the method further including: receiving data indicating a physiological parameter of a second rescuer at the rescue scene; determining that the physiological parameter of the second rescuer at the rescue scene is above the threshold; and in response to determining that the physiological parameter of the second rescuer at the rescue scene is above the threshold, refraining from causing the alarm to be output to the second rescuer.
18: The method of any of clauses 11 to 17, wherein comparing the physiological parameter of the patient to the threshold includes determining that chest compressions administered to the patient are ineffective, and wherein the alarm includes an instruction to change a frequency, depth, or location of the chest compressions.
19: The method of any of clauses 11 to 18, wherein comparing the physiological parameter of the patient to the threshold includes determining that assisted ventilation administered to the patient is ineffective, and wherein the alarm includes an instruction to check for a leak between a ventilation device and a face of the patient or to change a ventilation parameter of the assisted ventilation.
20: The method of any of clauses 11 to 19, wherein the parameter of the rescuer includes a physiological parameter corresponding to a probability of a level of stress of the rescuer.
21: A system, including: a wearable device configured to be worn by a rescuer, the wearable device including: a sensor configured to detect a parameter of the rescuer; a first transceiver configured to: transmit a communication signal indicating the parameter of the rescuer; and receive a communication signal indicating an alarm with an alarm type; and an output device configured to output the alarm with the alarm type; and a medical device, including: a measurement circuit configured to detect an electrocardiogram (ECG) of a patient; a second transceiver configured to: receive the communication signal indicating the parameter of the rescuer; and transmit the communication signal indicating the alarm type; and a processor configured to: determine, by analyzing the parameter, a type of treatment administered by the rescuer to the patient; identify an alarm type corresponding to the type of treatment administered by the rescuer to the patient; in response to identifying the alarm type, cause the second transceiver to transmit the communication signal indicating the alarm with the alarm type.
22: The system of clause 21, wherein the parameter includes an acceleration of the rescuer and the type of treatment includes chest compressions, and wherein the alarm type includes a cardiopulmonary resuscitation (CPR)-based alarm type corresponding to the chest compressions, the alarm instructing the rescuer to change a frequency, a depth, a location, or a duty cycle of the chest compressions.
23: The system of clause 21 or 22, wherein the parameter includes an acceleration of the rescuer and the type of treatment includes assisted ventilation, and wherein the alarm type includes a ventilation-based alarm type corresponding to the assisted ventilation, the alarm instructing the rescuer to change a rate of the assisted ventilation, to change a tidal volume of the assisted ventilation, to administer a medication, or to check for a leak between a ventilation device and an airway of the patient.
24: The system of any of clauses 21 to 23, wherein the parameter of the rescuer includes a blood pressure, a respiration rate, a temperature, an acceleration, a location, a vocal sound, or a pupil movement, and wherein the processor is configured to determine, by analyzing the parameter, the type of treatment administered by the rescuer to the patient by: identifying a treatment received by the patient; identifying a motion artifact in the parameter; determining that the motion artifact temporally corresponds to the treatment received by the patient.
25: A medical device, including: a sensor configured to detect a physiological parameter of a patient; a transceiver configured to: receive data indicating a parameter of a rescuer; and receiving a communication signal indicating an alarm; a processor configured to: generate the alarm by analyzing the physiological parameter of the patient; identify, by analyzing the parameter of the rescuer, that the rescuer is administering a treatment to the patient; in response to identifying that the rescuer is administering the treatment to the patient, cause the alarm to be output to the rescuer.
26: The medical device of clause 25, wherein the processor is configured to identify, by analyzing the parameter of the rescuer, that the rescuer is administering the treatment to the patient by determining that the rescuer is administering chest compressions to the patient, and wherein the alarm includes a cardiopulmonary resuscitation (CPR)-based alarm corresponding to the chest compressions.
27: The medical device of clause 25 or 26, wherein the parameter of the rescuer includes a blood pressure, a respiration rate, a temperature, an acceleration, a location, a vocal sound, a pupil movement, a pressure on a rescuer hand, or a short-rage communication-based proximity from a treatment device.
28: The medical device of clause 26, wherein the processor is configured to identify, by analyzing the parameter of the rescuer, that the rescuer is administering the treatment to the patient by determining that the rescuer is administering assisted ventilation to the patient, and wherein the alarm includes a ventilation-based alarm corresponding to the assisted ventilation.
29: The medical device of any of clauses 25 to 28, wherein the processor is further configured to cause the alarm to be output by: causing a device of the rescuer to output a projection indicating the alarm; causing the device of the rescuer to output a hologram indicating the alarm; or causing the device of the rescuer to output an augmented reality (AR) alarm.
30: The medical device of any of clauses 25 to 29, wherein the parameter includes an acceleration of the rescuer and a type of treatment administered by the rescuer to the patient includes chest compressions, and wherein an alarm type corresponding to the type of treatment administered by the rescuer to the patient includes a cardiopulmonary resuscitation (CPR)-based alarm type corresponding to the chest compressions, the alarm instructing the rescuer to change a frequency, a depth, a location, or a duty cycle of the chest compressions.
31: The medical device of any of clauses 25 to 30, wherein the parameter of the rescuer includes a blood pressure, a respiration rate, a temperature, an acceleration, a location, a vocal sound, or a pupil movement, and wherein the processor is configured to determine, by analyzing the parameter, a type of the treatment administered by the rescuer to the patient by: identifying the treatment received by the patient; identifying a motion artifact in the parameter; determining that the motion artifact temporally corresponds to the treatment received by the patient.
32: A method, including: detecting a physiological parameter of a patient; receiving data indicating a parameter of a rescuer; receiving a communication signal indicating an alarm; generating the alarm by analyzing the physiological parameter of the patient; identifying, by analyzing the parameter of the rescuer, that the rescuer is administering a treatment to the patient; and in response to identifying that the rescuer is administering a treatment to the patient, causing the alarm to be output to the rescuer.
33: The method of clause 32, further including: identifying, by analyzing the parameter of the rescuer, that the rescuer is administering the treatment to the patient by determining that the rescuer is administering chest compressions to the patient, wherein the alarm includes a cardiopulmonary resuscitation (CPR)-based alarm corresponding to the chest compressions.
34: The method of clause 32 or 33, wherein the parameter of the rescuer includes a blood pressure, a respiration rate, a temperature, an acceleration, a location, a vocal sound, a pupil movement, a pressure on a rescuer hand, or a short-rage communication-based proximity from a treatment device.
35: The method of any of clauses 32 to 34, wherein identifying that the rescuer is administering the treatment to the patient further includes: determining that the rescuer is administering assisted ventilation to the patient, and wherein the alarm includes a ventilation-based alarm corresponding to the assisted ventilation.
36: The method of any of clauses 32 to 35, further including: causing the alarm to be output by: causing a device of the rescuer to output a projection indicating the alarm; causing the device of the rescuer to output a hologram indicating the alarm; or causing the device of the rescuer to output an augmented reality (AR) alarm.
37: The method of any of clauses 32 to 36, wherein the parameter includes an acceleration of the rescuer, and wherein an alarm type corresponding to a type of treatment administered by the rescuer to the patient includes a cardiopulmonary resuscitation (CPR)-based alarm type corresponding to chest compressions administer by the rescuer, the alarm instructing the rescuer to change a frequency, a depth, a location, or a duty cycle of the chest compressions.
38: The method of any of clauses 32 to 37, wherein the parameter of the rescuer includes a blood pressure, a respiration rate, a temperature, an acceleration, a location, a vocal sound, or a pupil movement, further including: determining, by analyzing the parameter, a type of the treatment administered by the rescuer to the patient by: identifying the treatment received by the patient; identifying a motion artifact in the parameter; determining that the motion artifact temporally corresponds to the treatment received by the patient.
39: The method of any of clauses 32 to 38, further including: dividing alarms between devices of the rescuer and another rescuer, the alarms including the alarm being output to the rescuer and another alarm being output to the other rescuer.
40: The method of any of clauses 32 to 39, further including: identifying a role of the rescuer at a rescue scene, wherein generating the alarm is in response to identifying the role of the rescuer.

While the example clauses described above are described with respect to one particular implementation, it should be understood that, in the context of this document, the content of the example clauses can also be implemented via a method, device, system, computer-readable medium, and/or another implementation. Additionally, any one of examples 1-40 may be implemented alone or in combination with any other of the examples 1-40.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A system, comprising:
a wearable device configured to be worn by a rescuer, the wearable device comprising:
a sensor configured to detect a heart rate of the rescuer; and
a first transceiver configured to:
transmit a communication signal indicating the heart rate of the rescuer; and
receive a communication signal indicating an alarm; and
an output device configured to output the alarm to the rescuer; and
a medical device, comprising:
a measurement circuit configured to detect an electrocardiogram (ECG) of a patient;
a second transceiver configured to:
receive the communication signal indicating the heart rate of the rescuer; and
transmit the communication signal indicating the alarm; and
a processor configured to:
determine that the ECG is indicative of an arrhythmia;
in response to determining that the ECG is indicative of the arrhythmia, generate the alarm;
determine that the heart rate of the rescuer is below a threshold; and
in response to determining that the heart rate of the rescuer is below the threshold, cause the second transceiver to transmit the communication signal indicating the alarm.

2. The system of claim 1, wherein the output device is configured to output the alarm to the rescuer by:
outputting a projection indicating the alarm;
outputting a hologram indicating the alarm; or
outputting an augmented reality (AR) alarm.

3. The system of claim 1, wherein the wearable device comprises a smart watch, and
wherein the output device comprises a speaker configured to audibly output the alarm.

4. The system of claim 1, the heart rate being detected by the sensor at a first time, wherein the sensor is further configured to detect the heart rate of the rescuer at a second time,
wherein the first transceiver is further configured to transmit a communication signal indicating the heart rate of the rescuer at the second time,
wherein the second transceiver is further configured to receive the communication signal indicating the heart rate of the rescuer at the second time, and
wherein the processor is further configured to:
determine that the heart rate of the rescuer at the second time is above the threshold; and
in response to determining that the heart rate of the rescuer at the second time is above the threshold, cause the second transceiver to transmit a communication signal causing the wearable device to refrain from outputting the alarm.

5. A medical device, comprising:
a sensor configured to detect a physiological parameter of a patient;
a transceiver configured to receive data indicating a parameter of a rescuer;
a processor configured to:
generate an alarm by analyzing the physiological parameter of the patient;
compare the parameter of the rescuer to a threshold; and
in response to comparing the parameter of the rescuer to the threshold, cause the alarm to be output to the rescuer.

6. The medical device of claim 5, wherein the physiological parameter of the patient comprises a heart rate, an electrocardiogram (ECG), a respiration rate, capnograph, a blood pressure, a transthoracic impedance, a temperature, or a blood oxygenation.

7. The medical device of claim 5, wherein the physiological parameter of the patient comprises a first physiological parameter detected by the sensor at a first time, and
wherein the processor is further configured to:
determine that the first physiological parameter at a second time is above the threshold; and
in response to determining that the first physiological parameter at the second time is above the threshold, cause the transceiver to transmit a communication signal causing a wearable device configured to be worn by the rescuer to refrain from outputting the alarm.

8. The medical device of claim 5, wherein the parameter of the rescuer comprises a blood pressure, a respiration rate, a temperature, an acceleration, a location, a vocal sound, or a pupil movement.

9. The medical device of claim 5, wherein the parameter of the rescuer comprises a level of body movement detected by a motion sensor of a wearable device worn by the rescuer, a volume of a vocal sound detected by an audio recorder of the wearable device, or a pitch of the vocal sound.

10. The medical device of claim 5, wherein the parameter of the rescuer corresponds to a predicted stress level of the rescuer.

11. A method, comprising:
detecting a physiological parameter of a patient;
generating an alarm by analyzing the physiological parameter of the patient;
receiving data indicating a parameter of a rescuer;
comparing the parameter of the rescuer to a threshold;
in response to comparing the parameter of the rescuer to the threshold, causing the alarm to be output to the rescuer.

12. The method of claim 11, wherein the physiological parameter of the patient comprises a heart rate, an electrocardiogram (ECG), a respiration rate, a capnograph, a blood pressure, a transthoracic impedance, a temperature, or a blood oxygenation.

13. The method of claim 11, wherein generating the alarm by analyzing the physiological parameter of the patient comprises determining that the physiological parameter of the patient is indicative of an emergency medical condition.

14. The method of claim 11, wherein the parameter of the rescuer comprises a heart rate, a blood pressure, a respiration rate, a temperature, an acceleration, a location, a volume of a vocal sound, a pitch of the vocal sound, or a pupil movement.

15. The method of claim 11, wherein receiving the data indicating the parameter of the rescuer comprises:
receiving the data indicating the parameter of the rescuer from a portable computing device comprising smart glasses, a smart watch, or a recording device.

16. The method of claim 11, further comprising:
identifying a role of the rescuer at a rescue scene, the rescuer being one of multiple rescuers at the rescue scene; and
determining that the role of the rescuer is associated with the alarm,
wherein causing the alarm to be output to the rescuer is further in response to determining that the role of the rescuer is associated with the alarm.

17. The method of claim 11, the rescuer being a first rescuer at a rescue scene, the method further comprising:
receiving data indicating a physiological parameter of a second rescuer at the rescue scene;
determining that the physiological parameter of the second rescuer at the rescue scene is above the threshold; and
in response to determining that the physiological parameter of the second rescuer at the rescue scene is above the threshold, refraining from causing the alarm to be output to the second rescuer.

18. The method of claim 11, wherein comparing the physiological parameter of the patient to the threshold comprises determining that chest compressions administered to the patient are ineffective, and
wherein the alarm comprises an instruction to change a frequency, depth, or location of the chest compressions.

19. The method of claim 11, wherein comparing the physiological parameter of the patient to the threshold comprises determining that assisted ventilation administered to the patient is ineffective, and
wherein the alarm comprises an instruction to check for a leak between a ventilation device and a face of the patient or to change a ventilation parameter of the assisted ventilation.

20. The method of claim 11, wherein the parameter of the rescuer comprises a physiological parameter corresponding to a probability of a level of stress of the rescuer.
